(19) United States
(12) Patent Application Publication
(10) Pub. No.: US 2022/0220486 A1
(43) Pub. Date: Jul. 14, 2022

(54) COMBINATION TREATMENTS FOR CYSTIC FIBROSIS CHARACTERIZED BY A 3849 + 10KB C-TO-T CFTR MUTATION

(71) Applicants: YISSUM RESEARCH DEVELOPMENT COMPANY OF THE HEBREW UNIVERSITY OF JERUSALEM, Jerusalem (IL); SPLISENSE LTD., Jerusalem (IL)

(72) Inventors: Bat Sheva KEREM, Mevasseret Zion (IL); Efrat OZERI-GALAI, Jerusalem (IL); Yifat OREN, Jerusalem (IL); Ofra BARCHAD-AVITZUR, Jerusalem (IL)

(73) Assignees: YISSUM RESEARCH DEVELOPMENT COMPANY OF THE HEBREW UNIVERSITY OF JERUSALEM, Jerusalem (IL); SPLISENSE LTD., Jerusalem (IL)

(21) Appl. No.: 17/709,485

(22) Filed: Mar. 31, 2022

Related U.S. Application Data
(63) Continuation-in-part of application No. 17/607,908, filed on Nov. 1, 2021.
(60) Provisional application No. 62/843,469, filed on May 5, 2019.

(51) Int. Cl. C12N 15/113; A61K 31/4439; A61K 31/404; A61K 31/47; A61P 11/00

(52) U.S. Cl. CPC C12N 15/1138; A61K 31/4439; A61K 31/404; C12N 2310/11; A61P 11/00; C12N 2310/322; C12N 2320/31; A61K 31/47

(57) ABSTRACT

The present invention provides methods for treating Cystic Fibrosis (CF) and methods for suppressing the inclusion of a cryptic exon between exon 22 and 23 as a result of the mutation 3849+10 Kb C-to-T comprising the step of administering a pharmaceutical composition comprising synthetic oligonucleotides complementary to a region of the CFTR comprising the 3849+10 Kb C-to-T mutation oligonucleotides and a composition comprising one or more CFTR modifiers.

### CROSS-REFERENCE TO RELATED APPLICATIONS

This application is a continuation-in-part of U.S. patent application Ser. No. 17/607,908 titled “RESTORATION OF THE CFTR FUNCTION BY SPLICING MODULATION”, filed 1 Nov. 2021, which is a National Phase Application of PCT International Application No. PCT/IL2020/050495, International Filing Date 5 May 2020, claiming the benefit of U.S. Patent Application(s) No. 62/843,469, filed 5 May 2019, the contents of which are incorporated herein by reference in their entirety.

### SEQUENCE LISTING STATEMENT

The instant application contains a Sequence Listing which has been submitted electronically in ASCII format and is hereby incorporated by reference in its entirety. Said ASCII copy, created on Mar. 31, 2022, is named P-613881-US-SQL-31MAR22_ST25.txt and is 29 kilobytes in size.

### FIELD OF THE INVENTION

The present invention provides methods for treating Cystic Fibrosis (CF) and methods for suppressing the inclusion of a cryptic exon between exon 22 and 23 as a result of the mutation 3849+10 Kb C-to-T comprising the step of administering a pharmaceutical composition comprising synthetic oligonucleotides complementary to a region of the CFTR comprising the 3849+10 Kb C-to-T mutation oligonucleotides and a composition comprising one or more CFTR modifiers.

### BACKGROUND

Cystic fibrosis (CF) is a common, severe autosomal recessive disease caused by mutations in the CFTR gene. The CFTR gene encodes for a chloride channel responsible for chloride transport in epithelial cells. The major manifestations of CF are in the lungs, with more than 90% mortality related to the respiratory disease. The disease in the respiratory tract is linked to the insufficient CFTR function in the airway epithelium.

One of the most common CFTR mutations, which occurs in hundreds of CF patients worldwide, is the splicing mutation 3849+10 kb C-to-T, which leads to inclusion of an 84 base pair cryptic exon in the mature messenger RNA (mRNA) (denoted “intron 22 cryptic exon inclusion” mutation).

Anti-sense oligonucleotide (ASO) administration is one of the most promising therapeutic approaches for the treatment of genetic disorders caused by splicing mutations. ASOs are short synthetic molecules which can anneal to motifs predicted to be involved in the pre-mRNA splicing. ASO binding masks the targeted region and promote normal splicing. ASOs are highly specific for their targets and do not affect any other sequences in the cells.

There remains a constant need in the field of Cystic Fibrosis management for novel, potent therapeutics, designed to overcome the numerous mutations in the CFTR gene identified thus far, and restore CFTR function.

### SUMMARY

In some embodiments, the present invention provides a method for treating Cystic Fibrosis (CF) in a subject heterozygous for the 3849+10 Kb C-to-T mutation in the CFTR gene, comprising administering to said subject a composition comprising a therapeutically effective amount of a synthetic oligonucleotide complementary to a region of the CFTR comprising the 3849+10 Kb C-to-T mutation and a composition comprising a therapeutically effective amount of one or more CFTR modifiers, wherein said synthetic oligonucleotide suppresses the inclusion of intron 22 cryptic exon in the mature CFTR mRNA.

In some embodiments, the subject has a 3849+10 Kb C-to-T mutation in one allele and a F508del mutation in a second allele of the CFTR gene.

In some embodiments, the one or more CFTR modifiers comprises a CFTR-splicing-modulating agent, Translational Read-Through agent, a CFTR amplifier, a CFTR potentiator, or a CFTR corrector.

In some embodiments, the one or more CFTR modifiers comprises a different synthetic oligonucleotide molecule capable of suppressing intron 22 cryptic exon inclusion in the mature CFTR mRNA, Ataluren, ELX-02, QBW251, PTI-808, VX-561, VX-121, ivacaftor (VX-770), lumacaftor (VX-809), tezacaftor (VX-661), elexacaftor (VX-445), VX-659, VX-152, VX-440, ABBV-2222 (formerly GLPG2222), ABBV-191, ABBV-3067, ABBV-3221 (formerly GLPG-3221), FDL169, PTI-801, PTI-428, or a combination thereof.

In some embodiments, the composition comprising one or more CFTR modifiers comprises elexacaftor, tezacaftor, and ivacaftor.

In some embodiments, the synthetic oligonucleotide comprises: a phosphate-ribose backbone, a phosphate-deoxyribose backbone, a phosphorothioate-deoxyribose backbone, a 2′-O-methyl-phosphorothioate (2′ OMP) backbone, a phosphorodiamidate morpholino backbone, a peptide nucleic acid backbone, a 2-methoxyethyl phosphorothioate backbone, an alternating locked nucleic acid backbone, a phosphorothioate backbone, N3′-P5′ phosphoroamidates, 2′-deoxy-2′-fluoro-β-d-arabino nucleic acid, cyclohexene nucleic acid backbone nucleic acid, tricyclo-DNA (tcDNA) nucleic acid backbone, or a combination thereof.

In some embodiments, the synthetic oligonucleotide comprises a backbone with a 2′-Methoxy Ethyl (2′MOE) modification.

In some embodiments, the nucleotide sequence of said region of the CFTR comprising the 3849+10 Kb C-to-T mutation comprises SEQ ID NO: 37.

In some embodiments, the synthetic oligonucleotide molecule comprises a nucleotide sequence set forth in one of SEQ ID NOs: 1-25, and 41-44.

In some embodiments, the nucleotide sequence of said synthetic oligonucleotide molecule comprises the sequence as set forth in SEQ ID NO: 40.

In some embodiments, the treating comprises improving at least one clinical parameter of CF selected from the group consisting of: lung function, time to the first pulmonary exacerbation, change in weight, change in height, a change in Body Mass Index (BMI), change in the concentration of sweat chloride, number and/or duration of pulmonary exacerbations, total number of days of hospitalization for pulmonary exacerbations, and the need for antibiotic therapy for sinopulmonary signs or symptoms.

In some embodiments, the composition is administered via oral, nasal, inhalation, abdominal, subcutaneous, intra-peritoneal or intravenous administration.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 compares the effect of treatment with DMSO (control), TRIKAFTA® (elexacaftor/tezacaftor/ivacaftor), SPL84-23-1, and a combination of TRIKAFATA® and SPL84-23-1 on CFTR activity, tested using the Ussing Chamber assay. Data are presented as mean (±SEM) values of % of WT, calculated from the absolute values of ΔIscCFTRinh172(μA/cm²). Well differentiated primary HBE cells from a patient heterozygous for the 3849+10 kb C-to-T and F508del mutations were treated with the 200 nM of SPL84-23-1. 48 hours before experiments 3 μM VX661+1 μM VX-445 and 100 nM VX770 were added for the Trikafta treated filters. The level of WT was set according to the median ΔIscCFTRinh172 in HBE cultures from healthy WT/WT individuals.

FIG. 2 compares the effect of treatment with SYMDEKO® (a combination of tezacaftor and ivacaftor), a control ASO, and SPL84-23-1 tested using the Ussing Chamber assay. Data are presented as mean (±SEM) values of % of WT, calculated from the absolute values of ΔIscCFTRinh172(μA/cm²). Well differentiated primary HBE cells from a patient homozygous for the 3849 +10 kb C-to-T mutation were treated with the 200 nM of SPL84-23-1. 48 hours before experiments 3 μM VX661+100 nM VX770 were added for the Symdeko®-treated filters. The level of WT was set according to the median ΔIscCFTRinh172 in HBE cultures from healthy WT/WT individuals.

### DETAILED DESCRIPTION

In some embodiments, the present invention provides a method for treating Cystic Fibrosis (CF) in a subject having the 3849+10 Kb C-to-T mutation in the CFTR gene, comprising administering to said subject a composition comprising a therapeutically effective amount of a synthetic oligonucleotide complementary to a region of the CFTR comprising the 3849+10 Kb C-to-T mutation and a composition comprising a therapeutically effective amount of one or more CFTR modifiers, wherein said synthetic oligonucleotide suppresses the inclusion of intron 22 cryptic exon in the mature CFTR mRNA. In some embodiments, the subject is heterozygous for the 3849+10 Kb C-to-T mutation in the CFTR gene. In other embodiments, the subject is homozygous for the 3849+10 Kb C-to-T mutation in the CFTR gene.

In other embodiments, the present invention provides a method for suppressing the inclusion of intron 22 cryptic exon in the mature CFTR mRNA in a subject, comprising administering to said subject a composition comprising a therapeutically effective amount of a synthetic oligonucleotide complementary to a region of the CFTR comprising the 3849+10 Kb C-to-T mutation and a composition comprising a therapeutically effective amount of one or more CFTR modifiers. In some embodiments, the subject is heterozygous for the 3849+10 Kb C-to-T mutation in the CFTR gene. In other embodiments, the subject is homozygous for the 3849+10 Kb C-to-T mutation in the CFTR gene.

In some embodiments, the present invention provides methods of use for oligonucleotides and compositions comprising same, capable of binding to a CFTR pre-mRNA, thereby modulating splicing and restoring the function of the CFTR gene product. The present invention thus identifies sequences within the CFTR pre-mRNA which are targeted in order to modulate the splicing cascade of the CFTR pre-mRNA. Modulating CFTR pre-mRNA splicing, as demonstrated in the present invention, can avoid improper recognition of intron sequences as exons. As a result of the modulation of splicing, a functional CFTR protein is produced by an otherwise aberrant CFTR allele.

In some embodiments, the herein disclosed oligonucleotide for use in the methods as described herein is a synthetic oligonucleotide.

In some embodiments, artificial “anti-sense” polynucleotide molecules as described herein are able to target and bind predetermined sequences at the pre-mRNA molecule of the CFTR gene, and the binding modulates the splicing of the pre-mRNA molecule into mature mRNA, which subsequently translates into a functional CFTR protein. The targets within a CFTR pre-mRNA molecule are those discovered to be involved in splicing, either indirectly, by affecting the splicing of adjacent as well as remote sequences, or directly, by affecting their own splicing.

The present invention provides, in one aspect, a method comprising administering a synthetic oligonucleotide molecule, consisting of 17-21 consecutive bases that are complementary to a pre-mRNA transcript of a CFTR gene, wherein the synthetic oligonucleotide molecule at least partly suppresses the inclusion of intron 22 cryptic exon in the mature CFTR mRNA, increases the percentage of correctly spliced mature CFTR mRNA by at least about 10%; and decreases the level of aberrantly spliced mature CFTR mRNA by at least about 20%.

In another aspect, the present invention provides a method comprising administering a synthetic oligonucleotide molecule consisting of 17-21 consecutive bases that are complementary to a pre-mRNA transcript of a CFTR gene having a 3849+10 Kb C-to-T mutation, wherein the synthetic oligonucleotide molecule at least partly suppresses the inclusion of intron 22 cryptic exon in the mature CFTR mRNA, decreases the level of aberrantly spliced mature CFTR mRNA by at least about 20%.

The present invention also provides a method comprising administering a synthetic oligonucleotide molecule, consisting of 17-21 consecutive bases that are complementary to a pre-mRNA transcript of a CFTR gene having a 3849+10 Kb C-to-T mutation, wherein the synthetic oligonucleotide molecule at least partly suppresses the inclusion of intron 22 cryptic exon in the mature CFTR mRNA, increases the percentage of correctly spliced mature CFTR mRNA by at least about 10%; and decreases the level of aberrantly spliced mature CFTR mRNA by at least about 20%, wherein the oligonucleotide comprises a T-O-methyl-phosphorothioate backbone and/or 2′-Methoxy Ethyl (2′MOE) backbone. The phrase “suppress intron 22 cryptic exon inclusion” as used herein refers to lowering the occurrence of the addition of 84 nucleotides (SEQ ID NO: 35) found within intron 22 of the CFTR gene to the mature CFTR mRNA.

In some embodiments, “the percentage of correctly spliced mature CFTR mRNA” as used herein refers to the percentage of correctly spliced mature CFTR mRNA compared to the total mRNA within the same cells. In other embodiments, “the percentage of correctly spliced mature CFTR mRNA” is the percentage of correctly spliced mature CFTR mRNA compared to the amount of correctly spliced mature CFTR mRNA found in a healthy cell or subject not having a mutation in either allele of the CFTR gene. For example, an increase from 1% before treatment by the ASOs provided by the present invention to 11% after the treatment by the ASOs provided by the present invention is considered an increase of 10%. An increase from 10% before treatment by the ASOs provided by the present invention to 11% after the treatment by the ASOs provided by the present invention is considered an increase of 1%.

In certain embodiments, the CFTR transcript comprises a mutation that increases inclusion of an intron 22 cryptic exon. In certain embodiments, the mutation is a 3849+10 Kb C to T mutation. In some embodiments, the intron 22 cryptic exon comprises the sequence of SEQ ID NO: 35 or a fragment thereof.

In certain embodiments, the oligonucleotide molecule is complementary to a nucleotide sequence within SEQ ID NO: 37. In some embodiments, the oligonucleotide molecule is complementary to a nucleotide sequence not more than 1000, 900, 800, 700, 600, 500, 400, 300, 200 or 100 bases upstream of a mutation that increase inclusion of an intron 22 cryptic exon. Each possibility represents a separate embodiment of the invention. In some embodiments, the oligonucleotide molecule is complementary to a nucleotide sequence not more than 1000, 900, 800, 700, 600, 500, 400, 300, 200 or 100 bases downstream of a mutation that increase inclusion of an intron 22 cryptic exon. Each possibility represents a separate embodiment of the invention.

In some embodiments, the oligonucleotide has at least 75%, at least 80%, at least 85%, at least 90%, at least 95%, at least 99%, or 100% complementarity to a nucleotide sequence within SEQ ID NO: 37.

In certain embodiments, the oligonucleotide molecule is complementary to a nucleotide sequence corresponding to pre-mRNA molecule comprising a sequence about 100 base pairs before the intron 22 cryptic exon to about 100 base pairs after the intron 22 cryptic exon transcribed from a CFTR gene having a 3849+10 Kb C-to-T mutation. In certain embodiments, the oligonucleotide molecule is complementary to a nucleotide sequence within SEQ ID NO: 37.

The terms “complementary” or “complementarity” refer to the ability of nucleic acids, e.g., oligonucleotide, polynucleotide, etc., to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present invention, the ability to substitute a T is implied, unless otherwise stated.

In some embodiments, the oligonucleotide increases the percentage of correctly spliced mature CFTR mRNA by at least about 12%. In some embodiments, the oligonucleotide increases the percentage of correctly spliced mature CFTR mRNA by at least about 10%. In certain embodiments, the oligonucleotide molecule increases the percentage of correctly spliced mature CFTR mRNA by about 20%. In certain embodiments, the oligonucleotide molecule increases the percentage of correctly spliced mature CFTR mRNA by about 30%. In certain embodiments, the oligonucleotide molecule increases the percentage of correctly spliced mature CFTR mRNA by about 40%. In certain embodiments, the oligonucleotide molecule increases the percentage of correctly spliced mature CFTR mRNA by about 50%. In certain embodiments, the oligonucleotide molecule increases the percentage of correctly spliced mature CFTR mRNA by about 60%. In certain embodiments, the oligonucleotide molecule increases the percentage of correctly spliced mature CFTR mRNA by about 70%. In certain embodiments, the oligonucleotide molecule increases the percentage of correctly spliced mature CFTR mRNA by about 80%. In certain embodiments, the oligonucleotide molecule increases the percentage of correctly spliced mature CFTR mRNA by about 90%.

The phrase “increases the level of correctly spliced mature CFTR mRNA” as used herein refers to the increase in the level of correctly spliced mature CFTR mRNA after treatment by the ASOs provided by the present invention compared to the level before treatment or after mock-treatment. In certain embodiments, the increase in the level of correctly spliced mature CFTR mRNA is compared to mock-treatment by a control ASO. In certain embodiments, the control ASO consists of the nucleotide sequence in SEQ ID NO: 33.

In certain embodiments, the oligonucleotide molecule decreases the level of aberrantly spliced mature CFTR mRNA by about 30%. In certain embodiments, the oligonucleotide molecule decreases the level of aberrantly spliced mature CFTR mRNA by about 40%. In certain embodiments, the oligonucleotide molecule decreases the level of aberrantly spliced mature CFTR mRNA by about 50%. In certain embodiments, the oligonucleotide molecule decreases the level of aberrantly spliced mature CFTR mRNA by about 60%. In certain embodiments, the oligonucleotide molecule decreases the level of aberrantly spliced mature CFTR mRNA by about 70%. In certain embodiments, the oligonucleotide molecule decreases the level of aberrantly spliced mature CFTR mRNA by about 80%.

The phrase “decreases the level of aberrantly spliced mature CFTR mRNA” as used herein refers to the percentage decrease in the level of aberrantly spliced mature CFTR mRNA after treatment by the ASOs provided by the present invention compared to the level before treatment or after mock-treatment. In certain embodiments, the decrease in the level of aberrantly spliced mature CFTR mRNA is compared to mock-treatment by a control ASO. In certain embodiments, the control ASO consists of the nucleotide sequence in SEQ ID NO: 33. The phrase “increases the percentage of correctly spliced mature CFTR mRNA” as used herein refers to the percent increase in the ratio of correctly spliced to aberrantly spliced CFTR mRNA compared to untreated or mock-treatment.

In certain embodiments, the oligonucleotide molecule consists of 18 or 19 consecutive nucleotide bases. In certain embodiments, the oligonucleotide molecule consists of 18 consecutive nucleotide bases. In certain embodiments, the oligonucleotide molecule consists of 19 consecutive nucleotide bases. In certain embodiments, the oligonucleotide molecule consists of 20 consecutive nucleotide bases. In certain embodiments, the oligonucleotide molecule consists of 21 consecutive nucleotide bases.

In certain embodiments, the base is selected from the group consisting of adenine, guanine, cytosine, uracil and optionally thymine. In other certain embodiments, the base is selected from the group consisting of adenine, guanine, cytosine, and uracil. Each possibility represents a separate embodiment of the present invention.

In some embodiments, the oligonucleotide is chemically modified. In some embodiments, the chemical modification is a modification of a backbone of the oligonucleotide. In some embodiments, the chemical modification is a modification of a sugar of the oligonucleotide. In some embodiments, the chemical modification is a modification of a nucleobase of the oligonucleotide. In some embodiments, the chemical modification increases stability of the oligonucleotide in a cell. In some embodiments, the chemical modification increases stability of the oligonucleotide in vivo. In some embodiments, the chemical modification increases the oligonucleotide's ability to modulate splicing. In some embodiments, the chemical modification increases the oligonucleotide's ability to induce suppress the inclusion of intron 22 cryptic exon. In some embodiments, the chemical modification increases the half-life of the oligonucleotide. In some embodiments, the chemical modification inhibits polymerase extension from the 3′ end of the oligonucleotide. In some embodiments, the chemical modification inhibits recognition of the oligonucleotide by a polymerase. In some embodiments, the chemical modification inhibits double-strand trigged degradation. In some embodiments, the chemically modified oligonucleotide does not trigger nucleic acid double-stranded degradation upon binding a CFTR pre-mRNA. In some embodiments, the chemical modification inhibits RISC-mediated degradation. In some embodiments, the chemical modification inhibits RISC-mediated degradation or any parallel nucleic acid degradation pathway.

In certain embodiments, the consecutive nucleotide bases are linked by a backbone selected from the group consisting of a phosphate-ribose backbone, a phosphate-deoxyribose backbone, a 2′-O-methyl-phosphorothioate backbone, a phosphorodiamidate morpholino backbone, a peptide nucleic acid backbone, a 2-methoxyethyl phosphorothioate backbone, an alternating locked nucleic acid backbone, constrained ethyl backbone, and a phosphorothioate backbone. Each possibility represents a separate embodiment of the invention.

According to some embodiments, the oligonucleotide comprises a 2′-O-methyl-phosphorothioate backbone. According to other embodiments, the oligonucleotide comprises a 2′-Methoxy Ethyl (2′MOE) modification.

In some embodiments, the oligonucleotide comprises a 2′-O-methyl-phosphorothioate modification. In some embodiments, the oligonucleotide comprises a 2′MOE modification. In some embodiments, the modification is throughout the molecule. In some embodiments, the modification is at the 3′ end of the molecule. In some embodiments, the modification is at the 5′ end of the molecule. In some embodiments, the molecule comprises at least 1, 2, 3, 4, 5, 6, 7, 8, 9, 10, 11, 12, 13, 14, 15, 16, 17, 18, 19, 20, 21, 22, 23, 24 or 25 modifications. Each possibility represents a separate embodiment of the invention.

In some embodiments, the modified oligonucleotide is capable of completely restoring CFTR function compared to non-mutated CFTR. In other embodiments, the modified oligonucleotide is capable of restoring at least 40%, 50%, 60%, 70%, 75%, 80%, 85%, 90%, or 95% of CFTR function compared to non-mutated CFTR. Each possibility represents a separate embodiment of the invention.

In certain embodiments, the oligonucleotide molecule comprises or consists of a nucleotide sequence set forth in SEQ ID NO: 1. In certain embodiments, the oligonucleotide molecule comprises or consists of a nucleotide sequence set forth in SEQ ID NO: 2. In certain embodiments, the oligonucleotide molecule comprises or consists of a nucleotide sequence set forth in SEQ ID NO: 3. In certain embodiments, the oligonucleotide molecule comprises or consists of a nucleotide sequence set forth in SEQ ID NO: 4. In certain embodiments, the oligonucleotide molecule comprises or consists of a nucleotide sequence set forth in SEQ ID NO: 5. In certain embodiments, the oligonucleotide molecule comprises or consists of a nucleotide sequence set forth in SEQ ID NO: 6. In certain embodiments, the oligonucleotide molecule comprises or consists of a nucleotide sequence set forth in SEQ ID NO: 7. In certain embodiments, the oligonucleotide molecule comprises or consists of a nucleotide sequence set forth in SEQ ID NO: 8. In certain embodiments, the oligonucleotide molecule comprises or consists of a nucleotide sequence set forth in SEQ ID NO: 9. In certain embodiments, the oligonucleotide molecule comprises or consists of a nucleotide sequence set forth in SEQ ID NO: 10. In certain embodiments, the oligonucleotide molecule comprises or consists of a nucleotide sequence set forth in SEQ ID NO: 11. In certain embodiments, the oligonucleotide molecule comprises or consists of a nucleotide sequence set forth in SEQ ID NO: 12. In certain embodiments, the oligonucleotide molecule comprises or consists of a nucleotide sequence set forth in SEQ ID NO: 13. In certain embodiments, the oligonucleotide molecule comprises or consists of a nucleotide sequence set forth in SEQ ID NO: 14. In certain embodiments, the oligonucleotide molecule comprises or consists of a nucleotide sequence set forth in SEQ ID NO: 15. In certain embodiments, the oligonucleotide molecule comprises or consists of a nucleotide sequence set forth in SEQ ID NO: 16. In certain embodiments, the oligonucleotide molecule comprises or consists of a nucleotide sequence set forth in SEQ ID NO: 17. In certain embodiments, the oligonucleotide molecule comprises or consists of a nucleotide sequence set forth in SEQ ID NO: 18. In certain embodiments, the oligonucleotide molecule comprises or consists of a nucleotide sequence set forth in SEQ ID NO: 19. In certain embodiments, the oligonucleotide molecule comprises or consists of a nucleotide sequence set forth in SEQ ID NO: 20. In certain embodiments, the oligonucleotide molecule comprises or consists of a nucleotide sequence set forth in SEQ ID NO: 21. In certain embodiments, the oligonucleotide molecule comprises or consists of a nucleotide sequence set forth in SEQ ID NO: 22. In certain embodiments, the oligonucleotide molecule comprises or consists of a nucleotide sequence set forth in SEQ ID NO: 23. In certain embodiments, the oligonucleotide molecule comprises or consists of a nucleotide sequence set forth in SEQ ID NO: 24. In certain embodiments, the oligonucleotide molecule comprises or consists of a nucleotide sequence set forth in SEQ ID NO: 25. In certain embodiments, the oligonucleotide molecule comprises or consists of a nucleotide sequence set forth in SEQ ID NO: 40. In certain embodiments, the oligonucleotide molecule comprises or consists of a nucleotide sequence set forth in SEQ ID NO: 41. In certain embodiments, the oligonucleotide molecule comprises or consists of a nucleotide sequence set forth in SEQ ID NO: 42. In certain embodiments, the oligonucleotide molecule comprises or consists of a nucleotide sequence set forth in SEQ ID NO: 43. In certain embodiments, the oligonucleotide molecule comprises or consists of a nucleotide sequence set forth in SEQ ID NO: 44. In certain embodiments, the oligonucleotide molecule comprises or consists of a nucleotide sequence set forth in SEQ ID NO: 45. In certain embodiments, the oligonucleotide molecule comprises or consists of a nucleotide sequence set forth in SEQ ID NO: 46. In certain embodiments, the oligonucleotide molecule comprises or consists of a nucleotide sequence set forth in SEQ ID NO: 47. In certain embodiments, the oligonucleotide molecule comprises or consists of a nucleotide sequence set forth in SEQ ID NO: 48. In certain embodiments, the oligonucleotide molecule comprises or consists of a nucleotide sequence set forth in SEQ ID NO: 49. In certain embodiments, the oligonucleotide molecule comprises or consists of a nucleotide sequence set forth in SEQ ID NO: 50. In certain embodiments, the oligonucleotide molecule comprises or consists of a nucleotide sequence set forth in SEQ ID NO: 51. In certain embodiments, the oligonucleotide molecule comprises or consists of a nucleotide sequence set forth in SEQ ID NO: 52. In certain embodiments, the oligonucleotide molecule comprises or consists of a nucleotide sequence set forth in SEQ ID NO: 53. In certain embodiments, the oligonucleotide molecule comprises or consists of a nucleotide sequence set forth in SEQ ID NO: 54. In certain embodiments, the oligonucleotide molecule comprises or consists of a nucleotide sequence set forth in SEQ ID NO: 55. In certain embodiments, the oligonucleotide molecule comprises or consists of a nucleotide sequence set forth in SEQ ID NO: 56. In certain embodiments, the oligonucleotide molecule comprises or consists of a nucleotide sequence set forth in SEQ ID NO: 57. In certain embodiments, the oligonucleotide molecule comprises or consists of a nucleotide sequence set forth in SEQ ID NO: 58. In certain embodiments, the oligonucleotide molecule comprises or consists of a nucleotide sequence set forth in SEQ ID NO: 59. In certain embodiments, the oligonucleotide molecule comprises or consists of a nucleotide sequence set forth in SEQ ID NO: 60. In certain embodiments, the oligonucleotide molecule comprises or consists of a nucleotide sequence set forth in SEQ ID NO: 61. In certain embodiments, the oligonucleotide molecule comprises or consists of a nucleotide sequence set forth in SEQ ID NO: 62. In certain embodiments, the oligonucleotide molecule comprises or consists of a nucleotide sequence set forth in SEQ ID NO: 63. In certain embodiments, the oligonucleotide molecule comprises or consists of a nucleotide sequence set forth in SEQ ID NO: 64. In certain embodiments, the oligonucleotide molecule comprises or consists of a nucleotide sequence set forth in SEQ ID NO: 65. In certain embodiments, the oligonucleotide molecule comprises or consists of a nucleotide sequence set forth in SEQ ID NO: 66. In certain embodiments, the oligonucleotide molecule comprises or consists of a nucleotide sequence set forth in SEQ ID NO: 67. In certain embodiments, the oligonucleotide molecule comprises or consists of a nucleotide sequence set forth in SEQ ID NO: 68. In certain embodiments, the oligonucleotide molecule comprises or consists of a nucleotide sequence set forth in SEQ ID NO: 69. In certain embodiments, the oligonucleotide molecule comprises or consists of a nucleotide sequence set forth in SEQ ID NO: 70. In certain embodiments, the oligonucleotide molecule comprises or consists of a nucleotide sequence set forth in SEQ ID NO: 71.

In certain embodiments, the oligonucleotide molecule comprises a nucleotide sequence set forth in one of SEQ ID NO: 1 to SEQ ID NO: 5, and SEQ ID NO: 41. In certain embodiments, the oligonucleotide molecule consists of a nucleotide sequence set forth in SEQ ID NO: 1, in SEQ ID NO: 2, SEQ ID NO: 3, in SEQ ID NO: 4, in SEQ ID NO: 5, or in SEQ ID NO: 41. In certain embodiments, the oligonucleotide molecule comprises a nucleotide sequence set forth in SEQ ID NO: 40 or in SEQ ID NO: 1. Each possibility represents a separate embodiment of the invention.

In some embodiments, the oligonucleotide is specific to a CFTR pre-mRNA. As used herein, the term “specific” refers to both base pair specificity and also gene specificity. In some embodiments, the oligonucleotide is specific to the CFTR gene. In some embodiments, the oligonucleotide is specific to an intronic sequence of CFTR. In some embodiments, the oligonucleotide is specific to a cryptic exon in CFTR. In some embodiments, the oligonucleotide is specific to a nucleic acid sequence of intron 22 of CFTR. In some embodiments, the nucleic acid sequence of intron 22 is intron 22 cryptic exon of CFTR.

In some embodiments, the oligonucleotide binds the CFTR pre-mRNA with perfect complementarity. In some embodiments, the oligonucleotide does not bind any gene other than CFTR with perfect complementarity. In some embodiments, the oligonucleotide does not bind any gene other than CFTR with a complementarity of greater than 70, 75, 80, 85, 90, 95, 97, 99 or 100%. Each possibility represents a separate embodiment of the invention. In some embodiments, the oligonucleotide does not bind any gene other than CFTR with a complementarity of greater than 90%. In some embodiments, the oligonucleotide binds SEQ ID NO: 37 with perfect complementarity. In some embodiments, the oligonucleotide does not bind any sequence other than SEQ ID NO: 37 with perfect complementarity. In some embodiments, the oligonucleotide does not bind any sequence other than SEQ ID NO: 37 with complementarity of greater than 70, 75, 80, 85, 90, 95, 97, 99 or 100%. Each possibility represents a separate embodiment of the invention. In some embodiments, the oligonucleotide does not bind any sequence other than SEQ ID NO: 37 with a complementarity of greater than 90%. In some embodiments, the oligonucleotide does not bind with perfect complementarity to anywhere in the genome of a cell other than within CFTR. In some embodiments, the oligonucleotide does not bind with complementarity of greater than 70, 75, 80, 85, 90, 95, 97, 99 or 100% to anywhere in the genome of a cell other than within CFTR. Each possibility represents a separate embodiment of the invention. In some embodiments, the cell is a mammalian cell. In some embodiments, the mammal is a human.

In some embodiments, the oligonucleotide modulates expression of CFTR. In some embodiments, the oligonucleotide modulates splicing of CFTR. In some embodiments, the oligonucleotide modulates splicing, e.g., suppresses inclusion, of intron 22 cryptic exon of CFTR. In some embodiments, the oligonucleotide does not cause an off-target effect. In some embodiments, off-target is a target other than CFTR. In some embodiments, off-target is a target other than splicing, e.g., suppressing the inclusion, of intron 22 cryptic exon of CFTR. In some embodiments, the oligonucleotide does not substantially or significantly modulate expression of a gene other than CFTR. In some embodiments, the oligonucleotide does not substantially or significantly modulate splicing of a gene other than CFTR. In some embodiments, the oligonucleotide does not substantially or significantly modulate splicing of an exon other than intron 22 cryptic exon of CFTR. In some embodiments, substantial modulation of expression is a change in expression of at least 5, 10, 15, 20, 25, 30, 35, 40, 45 or 50%. Each possibility represents a separate embodiment of the invention. In some embodiments, substantial modulation of expression is a change in expression of at least 20%.

In some embodiments, an oligonucleotide as disclosed herein targets, complements, suppresses, or any combination thereof, the inclusion of intron 22 cryptic exon to the mature CFTR mRNA transcribed from a mutated allele of the CFTR gene. In some embodiments, an oligonucleotide as disclosed herein does not target, complement, suppresses, or any combination thereof, splicing modulation of CFTR pre-mRNA transcribed from a wild type allele of the CFTR gene. In some embodiments, an oligonucleotide as disclosed herein targets, complements, suppresses, or any combination thereof at least 2 fold more efficiently, at least 3 fold more efficiently, at least 5 fold more efficiently, at least 7 fold more efficiently, at least 10 fold more efficiently, at least 20 fold more efficiently, at least 50 fold more efficiently, or at least 100 fold more efficiently, the inclusion of intron 22 cryptic exon into the mature CFTR mRNA transcribed from a mutated allele of the CFTR gene compared to the wild type allele of the CFTR gene, or any value and range therebetween. Each possibility represents a separate embodiment of the invention. In some embodiments, an oligonucleotide as disclosed herein targets, complements, suppresses, or any combination thereof 2-10 fold more efficiently, 3-50 fold more efficiently, 5-100 fold more efficiently, 7-20 fold more efficiently, 2-40 fold more efficiently, 2-25 fold more efficiently, 50-150 fold more efficiently, or 2-100 fold more efficiently, the inclusion of intron 22 cryptic exon into the mature CFTR mRNA transcribed from a mutated allele of the CFTR gene compared to the wild type allele of the CFTR gene. Each possibility represents a separate embodiment of the invention.

In some embodiments, an oligonucleotide of the invention fully complements with a mutated allele of the CFTR gene. As used herein, the term “fully complements” refers to 100% hybridization, meaning the mutated CFTR allele and the oligonucleotide represent a reversed and complementary nucleic acid sequence versions of one another, as would be apparent to one of ordinary skill in the art of molecular biology. In some embodiments, an oligonucleotide of the invention partially complements with the wild type allele of the CFTR gene. As used herein, the term “partially” refers to any value or range lower than 100%. In some embodiments, the oligonucleotide of the invention and the wild type CFTR allele represent a reversed and complementary nucleic acid sequence version of one another which differ by at least one nucleotide, e.g., comprising at least one mismatched nucleotide.

In some embodiments, the oligonucleotide of the invention, and method of using same, provide the exclusion of a cryptic exon from the mature CFTR mRNA transcribed from a mutated allele of the CFTR gene. In some embodiments, the mature mRNA transcribed from the wild type allele is devoid of the cryptic exon.

In some embodiments, the cryptic exon is intron 22 cryptic exon. In some embodiments, the cryptic exon is 80-90 bases long.

In some embodiments, the oligonucleotide comprises an active fragment of any one of SEQ ID Nos.: 1-25 and 41-44.

In some embodiments, the oligonucleotide comprises an active fragment of any one of SEQ ID Nos.: 1-5 and 41.

As used herein, the term “active fragment” refers to a fragment that is 100% identical to a contiguous portion of the full nucleotide sequence of the oligonucleotide, providing that at least: 30%, 40%, 50%, 60%, 70%, 80% or 90% of the activity of the original oligonucleotide sequence is retained, or any value and range therebetween. Each possibility represents a separate embodiment of the present invention.

In some embodiments, the subject is heterozygous to the 3849 +10Kb C-to-T mutation. In some embodiments, a subject treated according to the method of the invention, comprises or is characterized by having a mixture of a wild type full-length and fully functional CFTR protein encoded from the wild type allele and a full-length and fully functional CFTR protein encoded from the pre-mRNA from which the inclusion of intron 22 cryptic exon was suppressed using the oligonucleotide of the invention. In some embodiments, the oligonucleotide of the invention does not reduce the level of the wild type full-length and fully functional CFTR protein in a subject, e.g., heterozygous to the mutation disclosed hereinabove.

The present invention further provides, in another aspect, a pharmaceutical composition comprising a synthetic oligonucleotide molecule as described above, and a pharmaceutically acceptable carrier.

The term “pharmaceutically acceptable carrier” as used herein refers to any of the standard pharmaceutical carriers known in the field such as sterile solutions, tablets, coated tablets, and capsules. Typically, such carriers contain excipients such as starch, milk, sugar, certain types of clay, gelatin, stearic acids, or salts thereof, magnesium or calcium stearate, talc, vegetable fats or oils, gums, glycols, or other known excipients. Such carriers may also include flavor and color additives or other ingredients. Examples of pharmaceutically acceptable carriers include, but are not limited to, the following: water, saline, buffers, inert, nontoxic solids (e.g., mannitol, talc). Compositions comprising such carriers are formulated by well-known conventional methods. Depending on the intended mode of administration and the intended use, the compositions may be in the form of solid, semi-solid, or liquid dosage forms, such, for example, as powders, granules, crystals, liquids, suspensions, liposomes, nano-particles, nano-emulsions, pastes, creams, salves, etc., and may be in unit-dosage forms suitable for administration of relatively precise dosages.

In certain embodiments, the pharmaceutical composition is formulated for oral administration. In certain embodiments, the pharmaceutical composition is formulated for nasal administration. In certain embodiments, the pharmaceutical composition is formulated for administration by inhalation. In certain embodiments, the pharmaceutical composition is formulated for abdominal administration. In certain embodiments, the pharmaceutical composition is formulated for subcutaneous administration. In certain embodiments, the pharmaceutical composition is formulated for intra-peritoneal administration. In certain embodiments, the pharmaceutical composition is formulated for intravenous administration.

In some embodiments, the pharmaceutical composition is formulated for systemic administration. In some embodiments, the pharmaceutical composition is formulated for administration to a subject. In some embodiments, the subject is a human subject. It will be understood by a skilled artisan that a pharmaceutical composition intended to administration to a subject should not have off-target effects, i.e. effects other than the intended therapeutic ones. In some embodiments, the pharmaceutical composition is devoid of a substantial effect on a gene other than CFTR. In some embodiments, the pharmaceutical composition is devoid of any substantial effect other than suppressing the inclusion of intron 22 cryptic exon to the mature CFTR. In some embodiments, a substantial effect is one with a phenotypic result. In some embodiments, a substantial effect is a deleterious effect. In some embodiments, deleterious is with respect to the health and/or wellbeing of the subject.

In some embodiments, the composition administered by inhalation. In some embodiments, the composition is an inhalation composition. in some embodiments, the composition is a pharmaceutical composition.

Being a long-known and well-studied disease, certain drugs and agents are known in the art for the treatment of Cystic Fibrosis patients. Administrating a synthetic polynucleotide molecule according to the present invention with one or more of these drugs may be beneficial in achieving significant therapeutic results.

In certain embodiments, the pharmaceutical composition further comprises one or more CFTR modifiers.

In some embodiments, the method further comprises administering to the subject a therapeutically effective amount of one or more CFTR modifiers.

In some embodiments, the CFTR modifier increases the duration of the CFTR gate being open, chloride flow through the CFTR gate, CFTR protein proper folding, the number of CFTR anchored to the cell membrane, or any combination thereof. Each possibility represents a separate embodiment of the invention.

In some embodiments, the modifier is selected from: potentiator, corrector, and amplifier.

As used herein, the term “potentiator” refers to any agent that increases the probability that a defective CFTR will be open and therefore allows chloride ions to pass through the channel pore.

As used herein, the term “corrector” refers to any agent that assists in proper CFTR channel folding so as to enable its trafficking to the cell membrane.

As used herein, the term “amplifier” refers to any agent that induces a cell to increase its CFTR protein production rates or yields, therefore resulting in an increased amount of the CFTR protein.

In certain embodiments, the CFTR modifier is selected from: a CFTR-splicing-modulating agent, Translational Read-Through agent, a CFTR amplifier, a CFTR potentiator and a CFTR corrector. In certain embodiments, the CFTR-splicing-modulating agent is a different synthetic oligonucleotide molecule capable of suppressing intron 22 cryptic exon inclusion in the mature CFTR mRNA; the Translational Read-Through agent is selected from the group consisting of 3-[5-(2-fluorophenyl)-1,2,4-oxadiazol-3-yl]benzoic acid (Ataluren) and ELX-02; the CFTR amplifier is PTI-428; the CFTR potentiator is selected from the group consisting of N-(2,4-Di-tert-butyl-5-hydroxyphenyl)-4-oxo-1,4-dihydroquinoline-3-carboxamide (Ivacaftor; VX-770), QBW251, PTI-808, ABBV-191, ABBV-2222, ABBV-3067, and VX-561 (deuterated ivacaftor); the CFTR potentiator is N-(2,4-Di-tert-butyl-5-hydroxyphenyl)-4-oxo-1,4-dihydroquinoline-3-carboxamide (Ivacaftor); or the CFTR corrector is selected from the group consisting of 3-{6-{[1-(2,2-difluoro-1,3-benzodioxol-5-yl)cyclopropanecarbonyl]amino}-3-methylpyridin-2-yl}benzoic acid (Lumacaftor; VX-809), 1-(2,2-difluoro-1,3-benzodioxol-5-yl)-{N}-[1-[(2-{R})-2,3-dihydroxypropyl]-6-fluoro-2-(1-hydroxy-2-methylpropan-2-yl)indol-5-yl]cyclopropane-1-carboxamide (Tezacaftor; VX-661), VX-659, elexacaftor (VX-445), VX-121, VX-152,VX-440, FDL169 and PTI-801.

In certain embodiments, the pharmaceutical composition comprises at least about 1 nM of the synthetic oligonucleotide molecule. In certain embodiments, the pharmaceutical composition comprises at least about 2.5 nM of the synthetic oligonucleotide molecule. In certain embodiments, the pharmaceutical composition comprises at least about 10 nM of the synthetic oligonucleotide molecule. In certain embodiments, the pharmaceutical composition comprises 2.5 nM to 10 nM of the synthetic oligonucleotide molecule.

In certain embodiments, the pharmaceutical composition comprises 1 nM to 1 μM of the synthetic oligonucleotide molecule. In certain embodiments, the pharmaceutical composition comprises 1 nM to 0.5 μM of the synthetic oligonucleotide molecule. In certain embodiments, the pharmaceutical composition comprises 1 nM to 100 nM of the synthetic oligonucleotide molecule.

The present invention further provides, in another aspect, a synthetic oligonucleotide molecule as described above, or a pharmaceutical composition as described above, for use in the modulation of splicing of a CFTR pre-mRNA transcribed from a CFTR gene having a 3849+10 Kb C-to-T mutation.

The phrase “modulation of splicing” as used herein refers to affecting a change in the level of any RNA or mRNA variant produced by the CFTR native pre-mRNA. For example, modulation may mean e.g. causing an increase or decrease in the level of abnormal CFTR mRNA, causing an increase or decrease in the level of normal, full-length CFTR mRNA, and/or causing an increase or decrease in the level of abnormal CFTR RNA or mRNA comprising a premature termination codon (non-sense codon). It is therefore evident that any change in ratio between certain CFTR splicing variants is also considered to be the result of splicing modulation. Each possibility represents a separate embodiment of the present invention. In certain embodiments, modulation means increasing the level of normal, full-length CFTR mRNA and/or decreasing the level of abnormal CFTR mRNA.

In certain embodiments, the use is for reducing the level of an mRNA molecule comprising the intron 22 cryptic exon. In certain embodiments, the use is for reducing the level of an mRNA molecule comprising the nucleotide sequence set forth in SEQ ID NO: 36. In certain embodiments, the use is for increasing the level of normal, full-length CFTR mRNA. In certain embodiments, the use is for increasing the level of an mRNA molecule comprising the nucleotide sequence set forth in SEQ ID NO: 34. In certain embodiments, the use is for correcting or improving chloride transport through the CFTR channel. In certain embodiments, the use is for increasing the production of functional CFTR protein. Each possibility represents a separate embodiment of the present invention.

The present invention further provides, in another aspect, a synthetic oligonucleotide molecule as described above, or a pharmaceutical composition as described above, for use in a method for improving at least one clinical parameter of Cystic Fibrosis.

The invention further provides, in another aspect, a method for improving at least one clinical parameter of Cystic Fibrosis in a patient in need thereof, comprising the step of administering a therapeutically effective amount of a synthetic polynucleotide molecule as described above to the patient.

The term “a therapeutically effective amount” as used herein refers to an amount necessary for improving at least one clinical parameter of Cystic Fibrosis or reducing the severity of at least one clinical parameter of Cystic Fibrosis in a patient. The therapeutically effective amounts may differ according to the patient's status, the synthetic polynucleotide molecule's administration route, excipient usage and co-usage of other active agents.

In certain embodiments, the clinical parameter is selected from the group consisting of lung function, time to the first pulmonary exacerbation, change in weight, change in height, a change in Body Mass Index (BMI), change in the concentration of sweat chloride, number and/or duration of pulmonary exacerbations, total number of days of hospitalization for pulmonary exacerbations, and the need for antibiotic therapy for sinopulmonary signs or symptoms. Each possibility represents a separate embodiment of the invention.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “condition” includes anatomic and physiological deviations from the normal that constitute an impairment of the normal state of the living animal or one of its parts, that interrupts or modifies the performance of the bodily functions.

As used herein, the terms “subject” or “individual” or “animal” or “patient” or “mammal,” refers to any subject, particularly a mammalian subject, for whom therapy is desired, for example, a human.

Being a genetic disease, Cystic Fibrosis currently cannot yet be cured, but its clinical manifestations and/or symptoms can be treated by the oligonucleotides of the present invention, for a marked increase and/or improvement in a patient's clinical status and quality of life.

The term “improving” as used herein refers to a favorable change, i.e., an increase or a decrease of at least 5%, at least 10%, at least 15%, at least 20%, at least 25%, at least 30%, at least 35%, at least 40%, at least 45%, or at least 50% in a clinical parameter of Cystic Fibrosis.

Different routes of AOs delivery have been examined in animal models and applied in clinical trials, chosen primarily according to the target tissue. For example, 2OMP was administrated to DMD patients (PRO-051) by local intramuscular injection (van Deutekom et al., 2007), and by abdominal subcutaneous injections (Goemans et al., 2011). 2OMP was also administrated to a SMA mouse model by intracerebroventricular injection (Williams et al., 2009; Hua et al., 2010). PMO was administrated to a DMD mouse model by intramuscular injection (Gebski, Mann, Fletcher, & Wilton, 2003), and repeated weekly intraperitoneal injections (Goyenvalle et al., 2010). PMO was also administrated to a SMA mouse model by intracerebroventricular injection (Porensky et al., 2012), and to DMD patients (AVI-4658) by local intramuscular injection (Kinali et al., 2009), or intravenously administration (Cirak et al., 2011; Mendell et al., 2013).

In certain embodiments, the method further comprises administering at least one additional anti-Cystic-Fibrosis agent to the patient. In certain such embodiments, the additional anti-Cystic-Fibrosis agent is selected from the group consisting of a CFTR-splicing-modulating agent, a CFTR potentiator and a CFTR corrector. Each possibility represents a separate embodiment of the present invention. In certain embodiments, the administration of the therapeutically effective amount of a synthetic polynucleotide molecule of the present invention and the administration of the at least one additional anti-Cystic-Fibrosis agent are independently oral, nasal, aerosol, inhalational, abdominal, subcutaneous, intra-peritoneal or intravenous administration. Each possibility represents a separate embodiment of the present invention. It should be understood that the selection of an administration route depends on the nature of the therapeutic agent and the site of its intended effect, and thus certain agents may be administrated via the same or different administration routes.

In certain embodiments, the administration of the synthetic oligonucleotide molecule or of the pharmaceutical composition is oral, nasal, inhalational, abdominal, subcutaneous, intra-peritoneal or intravenous administration.

In certain embodiments, the synthetic oligonucleotide molecule is administered in a concentration of at least about 1 nM. In certain embodiments, the synthetic oligonucleotide molecule is administered in a concentration of at least about 2.5 nM. In certain embodiments, the synthetic oligonucleotide molecule is administered in a concentration of at least about 10 nM. In certain embodiments, the synthetic oligonucleotide molecule is administered in a concentration of 2.5 nM to 10 nM.

The present invention further provides, in another aspect, the use of a synthetic oligonucleotide molecule as described above, or of a pharmaceutical composition as described above, in preparing a medicament.

In certain embodiments, the medicament is for treating or ameliorating a symptom of Cystic Fibrosis. In some embodiments, the medicament improves at least one clinical parameter of Cystic Fibrosis. According to some embodiments, the clinical parameter is selected from the group consisting of lung function, time to the first pulmonary exacerbation, change in weight, change in height, a change in Body Mass Index (BMI), change in the concentration of sweat chloride, number and/or duration of pulmonary exacerbations, total number of days of hospitalization for pulmonary exacerbations, and the need for antibiotic therapy for sinopulmonary signs or symptoms. Each possibility represents a separate embodiment of the invention.

The present invention further provides, in another aspect, a kit comprising a synthetic oligonucleotide molecule as described above.

In certain embodiments, the kit further comprises an additional anti-Cystic-Fibrosis agent.

In certain embodiments, the synthetic oligonucleotide and the additional anti-Cystic-Fibrosis agent are comprised in one pharmaceutical composition. In certain embodiments, the synthetic oligonucleotide and the additional anti-Cystic-Fibrosis agent are comprised in different pharmaceutical compositions. In certain embodiments, the synthetic oligonucleotide and the additional anti-Cystic-Fibrosis agent are independently formulated for oral, nasal, inhalation, abdominal, subcutaneous, or intra-peritoneal administration. Each possibility represents a different embodiment of the invention.

In certain embodiments, the synthetic oligonucleotide is in a concentration of at least about 1 nM. In certain embodiments, the synthetic oligonucleotide is in a concentration of at least about 2.5 nM. In certain embodiments, the synthetic oligonucleotide is in a concentration of at least about 10 nM. In certain embodiments, the synthetic oligonucleotide molecule is in a concentration of 2.5 nM to 10 nM.

The present invention further provides, in another aspect, a synthetic oligonucleotide molecule, consisting of 18-50 consecutive bases that are complementary to a pre-mRNA transcript of a CFTR gene having a 3849+10 Kb C-to-T mutation and at least partly suppresses the inclusion of intron 22 cryptic exon in the mature CFTR mRNA, comprising a nucleotide sequence set forth in one of SEQ ID NO: 1 to SEQ ID NO: 25, and SEQ ID NO: 41 to SEQ ID NO: 44.

The present invention further provides, in another aspect, a synthetic oligonucleotide molecule, consisting of 17-50 consecutive bases that are complementary to a pre-mRNA transcript of a CFTR gene having a 3849+10 Kb C-to-T mutation and at least partly suppresses the inclusion of intron 22 cryptic exon in the mature CFTR mRNA, comprising a nucleotide sequence set forth in one of SEQ ID NO: 1 to SEQ ID NO: 25 and SEQ ID NO: 40 to SEQ ID NO: 71.

In certain embodiments, the oligonucleotide molecule comprises or consists of a nucleotide sequence set forth in one of SEQ ID NO: 1 to SEQ ID NO: 10. In certain embodiments, the oligonucleotide molecule comprises or consists of a nucleotide sequence set forth in one of SEQ ID NO: 1 to SEQ ID NO: 5, and SEQ ID NO: 41. In certain embodiments, the oligonucleotide molecule comprises or consists of a nucleotide sequence set forth in SEQ ID NO: 1, in SEQ ID NO: 3, in SEQ ID NO: 4, in SEQ ID NO: 5, or SEQ ID NO: 41. In certain embodiments, the oligonucleotide molecule comprises or consists of a nucleotide sequence set forth in SEQ ID NO: 7.

In certain embodiments, the oligonucleotide molecule comprises a nucleotide sequence set forth in one of SEQ ID NO: 40 to SEQ ID NO: 71. In certain embodiments, the oligonucleotide molecule comprises a nucleotide sequence set forth in one of SEQ ID NO: 40 to SEQ ID NO: 48. In certain embodiments, the oligonucleotide molecule comprises a nucleotide sequence set forth in one of SEQ ID NO: 49 to SEQ ID NO: 53. In certain embodiments, the oligonucleotide molecule comprises a nucleotide sequence set forth in one of SEQ ID NO: 54 to SEQ ID NO: 58. In certain embodiments, the oligonucleotide molecule comprises a nucleotide sequence set forth in one of SEQ ID NO: 59 to SEQ ID NO: 63. In certain embodiments, the oligonucleotide molecule comprises a nucleotide sequence set forth in one of SEQ ID NO: 54 to SEQ ID NO: 71.

The present invention further provides, in another aspect, a synthetic oligonucleotide sequence consisting of 18-20 consecutive bases comprising the sequence GAUGGAAGA (SEQ ID NO: 38), wherein the synthetic oligonucleotide sequence is complementary to a pre-mRNA transcript of a CFTR gene having a 3849+10 Kb C-to-T mutation.

The present invention further provides, in another aspect, a synthetic oligonucleotide sequence consisting of 17-20 consecutive bases comprising the sequence GAUGGAAGA (SEQ ID NO: 38), wherein the synthetic oligonucleotide sequence is complementary to a pre-mRNA transcript of a CFTR gene having a 3849+10 Kb C-to-T mutation.

In certain embodiments, the oligonucleotide sequence is complementary to a nucleotide sequence within SEQ ID NO: 37. In certain embodiments, the oligonucleotide sequence is selected from the group consisting of: SEQ ID Nos: 1, 2, 4 and 7. In certain embodiments, the synthetic oligonucleotide sequence comprises the sequence CAACAGAUGGAAGA (SEQ ID NO: 39). In certain embodiments, the sequence is selected from the group consisting of: SEQ ID Nos: 1, 2 and 4.

In one embodiment, the present invention provides combined preparations. In one embodiment, “a combined preparation” defines especially a “kit of parts” in the sense that the combination partners as defined above can be dosed independently or by use of different fixed combinations with distinguished amounts of the combination partners i.e., simultaneously, concurrently, separately or sequentially. In some embodiments, the parts of the kit of parts can then, e.g., be administered simultaneously or chronologically staggered, that is at different time points and with equal or different time intervals for any part of the kit of parts. The ratio of the total amounts of the combination partners, in some embodiments, can be administered in the combined preparation.

In some embodiments, the kit of the invention comprises: at least one oligonucleotide; and at least one of: at least one CFTR modifier; or at least one CF drug, wherein the oligonucleotide is selected from SEQ ID Nos.: 1-25, and 41-44, and wherein the CFTR modifier is selected from: CFTR potentiator, CFTR corrector, and CFTR amplifier.

In some embodiments, the CF drug is an antibiotic drug, a bronchodilator, a corticosteroid, or any combination thereof.

Types and doses of CF drugs, such as an antibiotic, a bronchodilator, and a corticosteroid, would be apparent to one of ordinary skill in the art. Non-limiting examples of CF drugs, such as antibiotics include, but are not limited to, cloxacillin, dicloxacillin, cephalosporin, trimethoprim, sulfamethoxazole, erythromycin, amoxicillin, clavulanate, ampicillin, tetracycline, linezolid, tobramycin or aztreonam lysine, fluoroquinolone, gentamicin, and monobactam with antipseudomonal activity.

In some embodiments, the components of the kit disclosed above are sterile. As used herein, the term “sterile” refers to a state of being free from biological contaminants. Any method of sterilization is applicable and would be apparent to one of ordinary skill in the art.

In some embodiments, the components of the kit are packaged within a container.

In some embodiments, the container is made of a material selected from the group consisting of thin-walled film or plastic (transparent or opaque), paperboard-based foil, rigid plastic, metal (e.g., aluminum), glass, etc.

In some embodiments, the content of the kit is packaged, as described below, to allow for storage of the components until they are needed.

In some embodiments, some or all components of the kit may be packaged in suitable packaging to maintain sterility.

In some embodiments, the components of the kit are stored in separate containers within the main kit containment element e.g., box or analogous structure, may or may not be an airtight container, e.g., to further preserve the sterility of some or all of the components of the kit.

In some embodiments, the instructions may be recorded on a suitable recording medium or substrate. For example, the instructions may be printed on a substrate, such as paper or plastic, etc.

In some embodiments, the instructions may be present in the kit as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, etc. In other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

According to some embodiments, there is provided a method for producing a compound suitable for treating CF.

In some embodiments, the method comprises obtaining a compound that binds to intron 22 of the CFTR pre-mRNA. In some embodiments, the method comprises obtaining a compound that binds to SEQ ID NO: 37. In some embodiments, the method comprises assaying the inclusion of intron 22 cryptic exon in the mature CFTR mRNA in the presence of the obtained compound, and selecting at least one compound that suppresses the inclusion of intron 22 cryptic exon in the mature CFTR mRNA, thereby producing a compound suitable for treating CF.

In some embodiments, the compound is an oligonucleotide. In some embodiments, the oligonucleotide is an oligonucleotide as disclosed and as described herein.

Methods of assaying cryptic exon inclusion are common. Non-limiting examples of such methods include, but are not limited to, PCR, qPCR, gene sequencing, northern-blot, dot-blot, in situ hybridization, or others all of which would be apparent to one of ordinary skill in the art.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive “or”, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

Throughout this specification and claims, the word “comprise”, or variations such as “comprises” or “comprising”, indicate the inclusion of any recited integer or group of integers but not the exclusion of any other integer or group of integers.

As used herein, the term “consists essentially of”, or variations such as “consist essentially of” or “consisting essentially of”, as used throughout the specification and claims, indicate the inclusion of any recited integer or group of integers, and the optional inclusion of any recited integer or group of integers that do not materially change the basic or novel properties of the specified method, structure or composition.

As used herein, the terms “comprises”, “comprising”, “containing”, “having” and the like can mean “includes”, “including”, and the like; “consisting essentially of” or “consists essentially of” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises,” “comprising, “having” are/is interchangeable with “consisting”.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments unless the embodiment is inoperative without those elements.

The terms “comprises”, “comprising”, “includes”, “including”, “having” and their conjugates mean “including but not limited to”. This term encompasses the terms “consisting of” and “consisting essentially of”. As used herein, the singular form “a”, “an”, and “the” include plural references unless the context clearly dictates otherwise. For example, the term “a compound” or “at least one compound” may include a plurality of compounds, including mixtures thereof.

The following examples are meant to be construed as non-limiting to the scope of the invention and are to serve merely as illustrative embodiments.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Generally, the nomenclature used herein, and the laboratory procedures utilized in the present invention include chemical, molecular, biochemical, and cell biology techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); “Cell Biology: A Laboratory Handbook”, Volumes I-III Cellis, J. E., ed. (1994); The Organic Chemistry of Biological Pathways by John McMurry and Tadhg Begley (Roberts and Company, 2005); Organic Chemistry of Enzyme-Catalyzed Reactions by Richard Silverman (Academic Press, 2002); Organic Chemistry (6th Edition) by Leroy “Skip” G Wade; Organic Chemistry by T. W. Graham Solomons and, Craig Fryhle.

TABLE 1
Anti-Sense Oligonucleotides (ASOs).

| | SEQ ID | | Length |
|---|---|---|---|
| Name | NO: | Nucleotide sequence 5′→ 3′ | (nucleotides) |
| SPL84-23 | 1 | CUGCAACAGAUGGAAGACUC | 20 |
| SPL84-22 | 2 | CAACAGAUGGAAGACUCUU | 19 |
| SPL84-17 | 3 | CUCCAGAAAUCAAGAUGAC | 19 |
| SPL84-25 | 4 | UACUGCAACAGAUGGAAGA | 19 |
| SPL84-2 | 5 | AUCAAGAUGACAAGUCAACU | 20 |
| SPL84-16 | 6 | GUGGUCUCCAGAAAUCAAG | 19 |
| SPL84-21 | 7 | GAUGGAAGACUCUUGUAAU | 19 |
| SPL84-1 | 8 | CAAGAUGACAAGUCAACUGAA | 21 |
| SPL84-7 | 9 | GAAAUCAAGAUGACAAGUCAAC | 22 |
| SPL84-19 | 10 | ACCUUGUGGUCUCCAGAAA | 19 |
| SPL84-18 | 11 | CCAGAAAUCAAGAUGACAAG | 20 |
| SPL84-13 | 12 | CACCAUUUUAAUACUGCAACA | 21 |
| SPL84-24 | 13 | UGGAAGACUCUUGUAAUUAUU | 21 |
| SPL84-3 | 14 | GAUGACAAGUCAACUGAAAUU | 21 |
| SPL84-15 | 15 | CUUUCAGGGUGUCUUACUC | 19 |
| SPL84-14 | 16 | UCAGGGUGUCUUACUCACC | 19 |
| SPL84-20 | 17 | AUUACCUUGUGGUCUCCAGA | 20 |
| SPL84-5 | 18 | GACAAGUCAACUGAAAUUUAG | 21 |
| SPL84-8 | 19 | CAACUGAAAUUUAGAUCCACA | 21 |
| SPL84-6 | 20 | AGUCAACUGAAAUUUAGAUCC | 21 |
| SPL84-9 | 21 | GUGUCUUACUCACCAUUUUAA | 21 |
| SPL84-10 | 22 | GUGUCUUACUCACCAUUU | 18 |
| SPL84-12 | 23 | CUCACCAUUUUAAUACUGC | 19 |
| SPL84-4 | 24 | GACAAGUCAACUGAAAUU | 18 |
| SPL84-11 | 25 | CUUACUCACCAUUUUAAUAC | 20 |
| 5′ Kole | 26 | GUCUUACUCACCAUUUUA | 18 |
| 3′ Kole | 27 | CAAGUCAACUGAAAUUUAG | 19 |
| stop Kole | 28 | CUUGUAAUUAUUUUUACAU | 19 |
| ASO84-1 | 29 | AAAUCAAGAUGACAAGUCAACUGAA | 25 |
| ASO84-2 | 30 | CUUGUGGUCUCCAGAAAUCAAGAUG | 25 |
| ASO84-3 | 31 | AACAGAUGGAAGACUCUUGUAAUUA | 25 |
| ASO84-3 | 32 | UCAGGGUGUCUUACUCACCAUUUUA | 25 |
| Control | 33 | GACCACUUGCCACCCAUC | 18 |
| SPL84-23-1 | 40 | CUGCAACAGAUGGAAGACU | 19 |
| SPL84-23-2 | 41 | UGCAACAGAUGGAAGACUC | 19 |
| SPL84-23-3 | 42 | CUGCAACAGAUGGAAGAC | 18 |
| SPL84-23-4 | 43 | GCAACAGAUGGAAGACUC | 18 |
| SPL84-23-5 | 44 | UGCAACAGAUGGAAGACU | 18 |
| SPL84-23-6 | 45 | CUGCAACAGAUGGAAGA | 17 |
| SPL84-23-7 | 46 | CAACAGAUGGAAGACUC | 17 |
| SPL84-23-8 | 47 | UGCAACAGAUGGAAGAC | 17 |
| SPL84-23-9 | 48 | GCAACAGAUGGAAGACU | 17 |
| | 49 | CUCCAGAAAUCAAGAUGA | 18 |
| | 50 | UCCAGAAAUCAAGAUGAC | 18 |
| | 51 | CUCCAGAAAUCAAGAUG | 17 |
| | 52 | CCAGAAAUCAAGAUGAC | 17 |
| | 53 | UCCAGAAAUCAAGAUGA | 17 |
| | 54 | CAACAGAUGGAAGACUCU | 18 |
| | 55 | AACAGAUGGAAGACUCUU | 18 |
| | 56 | ACAGAUGGAAGACUCUU | 17 |
| | 57 | AACAGAUGGAAGACUCU | 17 |
| | 58 | UACUGCAACAGAUGGAAG | 18 |
| | 59 | ACUGCAACAGAUGGAAGA | 18 |
| | 60 | UACUGCAACAGAUGGAA | 17 |
| | 61 | ACUGCAACAGAUGGAAG | 17 |
| | 62 | AUCAAGAUGACAAGUCAAC | 19 |
| | 63 | UCAAGAUGACAAGUCAACU | 19 |
| | 64 | AUCAAGAUGACAAGUCAA | 18 |
| | 65 | CAAGAUGACAAGUCAACU | 18 |
| | 66 | UCAAGAUGACAAGUCAAC | 18 |
| | 67 | AUCAAGAUGACAAGUCA | 17 |
| | 68 | AAGAUGACAAGUCAACU | 17 |
| | 69 | UCAAGAUGACAAGUCAA | 17 |
| | 70 | CAAGAUGACAAGUCAAC | 17 |
| SPL84-26 | 71 | AAUUAUUUUUCAUUACCUUG | 20 |

TABLE 2
CFTR-related sequences (all in sense orientation).

| | SEQ ID | |
|---|---|---|
| Name | NO: | Chr. 7 position |
| Mature CFTR mRNA | 34 | aauuggaagc aaaugacauc acagcagguc agagaaaaag gguugagcgg |
| | | caggcacccagaguaguagg ucuuuggcau uaggagcuug agcccagacg |
| | | gcccuagcag ggaccccagc gcccgagaga ccaugcagag gucgccucug |
| | | gaaaaggcca gcguugucuc caaacuuuuu uucagcugga ccagaccaau |
| | | uuugaggaaa ggauacagac agcgccugga auugucagac auauaccaaa |
| | | ucccuucugu ugauucugcu gacaaucuau cugaaaaauu gaaagagaa |
| | | ugggauagag agcuggcuuc aaagaaaaau ccuaaacuca uuaaugcccu |
| | | ucggcgaugu uuuuucugga gauuuauguu cuauggaauc uuuuuauauu |
| | | uaggggaagu caccaaagca guacagccuc ucuuacuggg aagaaucaua |
| | | gcuuccuaug acccggauaa caaggaggaa cgcucuaucg cgauuuaucu |
| | | aggcauaggc uuaugccuuc ucuuuauugu gaggacacug cuccuacacc |
| | | cagccauuuu uggccuucau cacauuggaa ugcagaugag aauagcuaug |
| | | uuuaguuuga uuuauaagaa gacuuuaaag cugucaagcc guguucuaga |
| | | uaaaauaagu auuggacaac uuguuagucu ccuuuccaac aaccugaaca |
| | | aauuugauga aggacuugca uuggcacauu ucguguggau cgcuccuuug |
| | | caaguggcac uccucauggg gcuaaucugg gaguuguuac aggcgucugc |
| | | cuucugugga cuugguuucc ugauaguccu ugcccuuuuu caggcugggc |
| | | uagggagaau gaugaugaag uacagagauc agagagcugg gaagaucagu |
| | | gaaagacuug ugauuaccuc agaaaugauu gaaaauaucc aaucuguuaa |
| | | ggcauacugc ugggaagaag caauggaaaa aaugauugaa aacuuaagac |
| | | aaacagaacu gaaacugacu cggaaggcag ccuaugugag auacuucaau |
| | | agcucagccu ucuucuucuc aggguucuuu gugguguuuu uaucugugcu |
| | | ucccuaugca cuaaucaaag gaaucauccu ccggaaaaua uucaccacca |
| | | ucucauucug cauuguucug cgcauggcgg ucacucggca auuucccugg |
| | | gcuguacaaa caugguauga cucucuugga gcaauaaaca aaauacagga |
| | | uuucuuacaa aagcaagaau auaagacauu ggaauauaac uuaacgacua |
| | | cagaaguagu gauggagaau guaacagccu ucugggagga gggauuuggg |
| | | gaauuauuug agaaagcaaa acaaaacaau aacaauagaa aaacuucuaa |
| | | uggugaugac agccucuucu ucaguaauuu cucacuucuu gguacuccug |
| | | uccugaaaga uauuaauuuc aagauagaaa gaggacaguu guuggcgguu |
| | | gcuggaucca cuggagcagg caagacuuca cuucuaaugg ugauuauggg |
| | | agaacuggag ccuucagagg guaaaauuaa gcacagugga agaauuucau |
| | | ucuguucuca guuuuccugg auuaugccug gcaccauuaa agaaaauauc |
| | | aucuuuggug uuuccuauga ugaauauaga uacagaagcg ucaucaaagc |
| | | augccaacua gaagaggaca ucuccaaguu ugcagagaaa gacaauauag |
| | | uucuuggaga agguggaauc acacugagug gaggucaacg agcaagaauu |
| | | ucuuuagcaa gagcaguaua caaagaugcu gauuuguauu uauuagacuc |
| | | uccuuuugga uaccuagaug uuuuaacaga aaaagaaaua uuugaaagcu |
| | | gugucuguaa acugauggcu aacaaaacua ggauuuuggu cacuucuaaa |
| | | auggaacauu uaaagaaagc ugacaaaaua uuaauuuugc augaagguag |
| | | cagcuauuuu uaugggacau uuucagaacu ccaaaaucua cagccagacu |
| | | uuagcucaaa acucauggga ugugauucuu ucgaccaauu uagugcagaa |
| | | agaagaaauu caauccuaac ugagaccuua caccguuucu cauuagaagg |
| | | agaugcuccu gucuccugga cagaaacaaa aaaacaaucu uuuaaacaga |
| | | cuggagaguu uggggaaaaa aggaagaauu cuauucucaa uccaaucaac |
| | | ucuauacgaa aauuuuccau ugugcaaaag acucccuuac aaaugaaugg |
| | | caucgaagag gauucugaug agccuuuaga gagaaggcug uccuuaguac |
| | | cagauucuga gcagggagag gcgauacugc cucgcaucag cgugaucagc |
| | | acuggcccca cgcuucaggc acgaaggagg cagucugucc ugaaccugau |
| | | gacacacuca guuaaccaag gucagaacau ucaccgaaag acaacagcau |
| | | ccacacgaaa agugucacug gccccucagg caaacuugac ugaacuggau |
| | | auauauucaa gaagguuauc ucaagaaacu ggcuuggaaa uaagugaaga |
| | | aauuaacgaa gaagacuuaa aggagugcuu uuuugaugau auggagagca |
| | | uaccagcagu gacuacaugg aacacauacc uucgauauau uacuguccac |
| | | aagagcuuaa uuuuugugcu aauuuggugc uuaguaauuu uucuggcaga |
| | | gguggcugcu ucuuugguug ugcuguggcu ccuuggaaac acuccucuuc |
| | | aagacaaagg gaauaguacu cauaguagaa auaacagcua ugcagugauu |
| | | aucaccagca ccaguucgua uuauguguuu uacauuuacg ugggaguagc |
| | | cgacacuuug cuugcuaugg gauucuucag aggucuacca cuggugcaua |
| | | cucuaaucac agugucgaaa auuuuacacc acaaaauguu acauucuguu |
| | | cuucaagcac cuaugucaac ccucaacacg uugaaagcag gugggauucu |
| | | uaauagauuc uccaaagaua uagcaauuuu ggaugaccuu cugccucuua |
| | | ccauauuuga cuucauccag uuguuauuaa uugugauugg agcuauagca |
| | | guugucgcag uuuuacaacc cuacaucuuu guugcaacag ugccagugau |
| | | aguggcuuuu auuauguuga gagcauauuu ccuccaaacc ucacagcaac |
| | | ucaaacaacu ggaaucugaa ggcaggaguc caauuuucac ucaucuuguu |
| | | acaagcuuaa aaggacuaug gacacuucgu gccuucggac ggcagccuua |
| | | cuuugaaacu cuguuccaca aagcucugaa uuuacauacu gccaacuggu |
| | | ucuuguaccu gucaacacug cgcugguucc aaaugagaau agaaaugauu |
| | | uuugucaucu ucuucauugc uguuaccuuc auuuccauuu uaacaacagg |
| | | agaaggagaa ggaagaguug guauuauccu gacuuuagcc augaauauca |
| | | ugaguacauu gcagugggcu guaaacucca gcauagaugu ggauagcuug |
| | | augcgaucug ugagccgagu cuuuaaguuc auugacaugc caacagaagg |
| | | uaaaccuacc aagucaacca aaccauacaa gaauggccaa cucucgaaag |
| | | uuaugauuau ugagaauuca cacgugaaga aagaugacau cuggcccuca |
| | | gggggccaaa ugacugucaa agaucucaca gcaaaauaca cagaaggugg |
| | | aaaugccaua uuagagaaca uuuccuucuc aauaaguccu ggccagaggg |
| | | ugggccucuu gggaagaacu ggaucaggga agaguacuuu guuaucagcu |
| | | uuuuugagac uacugaacac ugaaggagaa auccagaucg augguguguc |
| | | uugggauuca auaacuuugc aacaguggag gaaagccuuu ggagugauac |
| | | cacagaaagu auuuauuuuu ucuggaacau uuagaaaaaa cuuggauccc |
| | | uaugaacagu ggagugauca agaaauaugg aaaguugcag augagguugg |
| | | gcucagaucu gugauagaac aguuuccugg gaagcuugac uuuguccuug |
| | | uggauggggg cuguguccua agccauggcc acaagcaguu gaugugcuug |
| | | gcuagaucug uucucaguaa ggcgaagauc uugcugcuug augaacccag |
| | | ugcucauuug gauccaguaa cauaccaaau aauuagaaga acucuaaaac |
| | | aagcauuugc ugauugcaca guaauucucu gugaacacag gauagaagca |
| | | augcuggaau gccaacaauu uuuggucaua gaagagaaca aagugcggca |
| | | guacgauucc auccagaaac ugcugaacga gaggagccuc uuccggcaag |
| | | ccaucagccc cuccgacagg gugaagcucu uuccccaccg gaacucaagc |
| | | aagugcaagu cuaagcccca gauugcugcu cugaaagagg agacagaaga |
| | | agaggugcaa gauacaaggc uuuagagagc agcauaaaug uugacauggg |
| | | acauuugcuc auggaauugg agcucguggg acagucaccu cauggaauug |
| | | gagcucgugg aacaguuacc ucugccucag aaaacaagga ugaauuaagu |
| | | uuuuuuuuaa aaaagaaaca uuugguaagg gaauugagg acacugauau |
| | | gggucuugau aaauggcuuc cuggcaauag ucaaauugug ugaaagguac |
| | | uucaaauccu ugaagauuua ccacuugugu uuugcaagcc agauuuuccu |
| | | gaaaacccuu gccaugugcu aguaauugga aaggcagcuc uaaaugucaa |
| | | ucagccuagu ugaucagcuu auugucuagu gaaacucguu aauuuguagu |
| | | guuggagaag aacugaaauc auacuucuua ggguuaugau uaaguaauga |
| | | uaacuggaaa cuucagcggu uuauauaagc uuguauuccu uuuucucucc |
| | | ucuccccaug auguuuagaa acacaacuau auuguuugcu aagcauucca |
| | | acuaucucau uuccaagcaa guauuagaau accacaggaa ccacaagacu |
| | | gcacaucaaa auaugcccca uucaacaucu agugagcagu caggaaagag |
| | | aacuuccaga uccuggaaau caggguuagu auuguccagg ucuaccaaaa |
| | | aucucaauau uucagauaau cacaauacau cccuuaccug ggaaagggcu |
| | | guuauaaucu uucacagggg acaggauggu ucccuugaug aagaaguuga |
| | | uaugccuuuu cccaacucca gaaagugaca agcucacaga ccuuugaacu |
| | | agaguuuagc uggaaaagua uguuagugca aauugucaca ggacagcccu |
| | | ucuuuccaca gaagcuccag guagagggug uguaaguaga uaggccaugg |
| | | gcacuguggg uagacacaca ugaaguccaa gcauuuagau guauagguug |
| | | auggugguau guuuucaggc uagauguaug uacuucaugc ugucuacacu |
| | | aagagagaau gagagacaca cugaagaagc accaaucaug aauuaguuuu |
| | | auaugcuucu guuuuauaau uuugugaagc aaaauuuuuu cucuaggaaa |
| | | uauuuauuuu aauaauguuu caaacauaua uaacaaugcu guauuuuaaa |
| | | agaaugauua ugaauuacau uuguauaaaa uaauuuuuau auuugaaaua |
| | | uugacuuuuu auggcacuag uauuucuaug aaauauuaug uuaaaacugg |
| | | gacaggggag aaccuagggu gauauuaacc aggggccaug aaucaccuuu |
| | | uggucuggag ggaagccuug gggcugaugc aguuguugcc cacagcugua |
| | | ugauucccag ccagcacagc cucuuagaug caguucugaa gaagauggua |
| | | ccaccagucu gacuguuucc aucaagggua cacugccuuc ucaacuccaa |
| | | acugacucuu aagaagacug cauuauauuu auuacuguaa gaaaauauca |
| | | cuugucaaua aaauccauac auuuguguga aa |
| Intron 22 cryptic exon * | 35 | TTGACTTGTCATCTTGATTTCTGGAGACCACAAGGTAATGA |
| | | AAAATAATTACAAGAGTCTTCCATCTGTTGCAGTATTAAAA |
| | | TG |
| Exons 1-27 + cryptic | 36 | aauuggaagc aaaugacauc acagcagguc agagaaaaag gguugagcgg |
| exon 22 | | caggcaccca gaguaguagg ucuuuggcau uaggagcuug agcccagacg |
| | | gcccuagcag ggaccccagc gcccgagaga ccaugcagag gucgccucug |
| | | gaaaaggcca gcguugucuc caaacuuuuu uucagcugga ccagaccaau |
| | | uuugaggaaa ggauacagac agcgccugga auugucagac auauaccaaa |
| | | ucccuucugu ugauucugcu gacaaucuau cugaaaaauu ggaaagagaa |
| | | ugggauagag agcuggcuuc aaagaaaaau ccuaaacuca uuaaugcccu |
| | | ucggcgaugu uuuuucugga gauuuauguu cuauggaauc uuuuuauauu |
| | | uaggggaagu caccaaagca guacagccuc ucuuacuggg aagaaucaua |
| | | gcuuccuaug acccggauaa caaggaggaa cgcucuaucg cgauuuaucu |
| | | aggcauaggc uuaugccuuc ucuuuauugu gaggacacug cuccuacacc |
| | | cagccauuuu uggccuucau cacauuggaa ugcagaugag aauagcuaug |
| | | uuuaguuuga uuuauaagaa gacuuuaaag cugucaagcc guguucuaga |
| | | uaaaauaagu auuggacaac uuguuagucu ccuuuccaac aaccugaaca |
| | | aauuugauga aggacuugca uuggcacauu ucguguggau cgcuccuuug |
| | | caaguggcac uccucauggg gcuaaucugg gaguuguuac aggcgucugc |
| | | cuucugugga cuugguuucc ugauaguccu ugcccuuuuu caggcugggc |
| | | uagggagaau gaugaugaag uacagagauc agagagcugg gaagaucagu |
| | | gaaagacuug ugauuaccuc agaaaugauu gaaaauaucc aaucuguuaa |
| | | ggcauacugc ugggaagaag caauggaaaa aaugauugaa aacuuaagac |
| | | aaacagaacu gaaacugacu cggaaggcag ccuaugugag auacuucaau |
| | | agcucagccu ucuucuucuc aggguucuuu gugguguuuu uaucugugcu |
| | | ucccuaugca cuaaucaaag gaaucauccu ccggaaaaua uucaccacca |
| | | ucucauucug cauuguucug cgcauggcgg ucacucggca auuucccugg |
| | | gcuguacaaa caugguauga cucucuugga gcaauaaaca aaauacagga |
| | | uuucuuacaa aagcaagaau auaagacauu ggaauauaac uuaacgacua |
| | | cagaaguagu gauggagaau guaacagccu ucugggagga gggauuuggg |
| | | gaauuauuug agaaagcaaa acaaaacaau aacaauagaa aaacuucuaa |
| | | uggugaugac agccucuucu ucaguaauuu cucacuucuu gguacuccug |
| | | uccugaaaga uauuaauuuc aagauagaaa gaggacaguu guuggcgguu |
| | | gcuggaucca cuggagcagg caagacuuca cuucuaaugg ugauuauggg |
| | | agaacuggag ccuucagagg guaaaauuaa gcacagugga agaauuucau |
| | | ucuguucuca guuuuccugg auuaugccug gcaccauuaa agaaaauauc |
| | | aucuuuggug uuuccuauga ugaauauaga uacagaagcg ucaucaaagc |
| | | augccaacua gaagaggaca ucuccaaguu ugcagagaaa gacaauauag |
| | | uucuuggaga agguggaauc acacugagug gaggucaacg agcaagaauu |
| | | ucuuuagcaa gagcaguaua caaagaugcu gauuuguauu uauuagacuc |
| | | uccuuuugga uaccuagaug uuuuaacaga aaaagaaaua uuugaaagcu |
| | | gugucuguaa acugauggcu aacaaaacua ggauuuuggu cacuucuaaa |
| | | auggaacauu uaaagaaagc ugacaaaaua uuaauuuugc augaagguag |
| | | cagcuauuuu uaugggacau uuucagaacu ccaaaaucua cagccagacu |
| | | uuagcucaaa acucauggga ugugauucuu ucgaccaauu uagugcagaa |
| | | agaagaaauu caauccuaac ugagaccuua caccguuucu cauuagaagg |
| | | agaugcuccu gucuccugga cagaaacaaa aaaacaaucu uuuaaacaga |
| | | cuggagaguu uggggaaaaa aggaagaauu cuauucucaa uccaaucaac |
| | | ucuauacgaa aauuuuccau ugugcaaaag acucccuuac aaaugaaugg |
| | | caucgaagag gauucugaug agccuuuaga gagaaggcug uccuuaguac |
| | | cagauucuga gcagggagag gcgauacugc cucgcaucag cgugaucagc |
| | | acuggcccca cgcuucaggc acgaaggagg cagucugucc ugaaccugau |
| | | gacacacuca guuaaccaag gucagaacau ucaccgaaag acaacagcau |
| | | ccacacgaaa agugucacug gccccucagg caaacuugac ugaacuggau |
| | | auauauucaa gaagguuauc ucaagaaacu ggcuuggaaa uaagugaaga |
| | | aauuaacgaa gaagacuuaa aggagugcuu uuuugaugau auggagagca |
| | | uaccagcagu gacuacaugg aacacauacc uucgauauau uacuguccac |
| | | aagagcuuaa uuuuugugcu aauuuggugc uuaguaauuu uucuggcaga |
| | | gguggcugcu ucuuugguug ugcuguggcu ccuuggaaac acuccucuuc |
| | | aagacaaagg gaauaguacu cauaguagaa auaacagcua ugcagugauu |
| | | aucaccagca ccaguucgua uuauguguuu uacauuuacg ugggaguagc |
| | | cgacacuuug cuugcuaugg gauucuucag aggucuacca cuggugcaua |
| | | cucuaaucac agugucgaaa auuuuacacc acaaaauguu acauucuguu |
| | | cuucaagcac cuaugucaac ccucaacacg uugaaagcag gugggauucu |
| | | uaauagauuc uccaaagaua uagcaauuuu ggaugaccuu cugccucuua |
| | | ccauauuuga cuucauccag uuguuauuaa uugugauugg agcuauagca |
| | | guugucgcag uuuuacaacc cuacaucuuu guugcaacag ugccagugau |
| | | aguggcuuuu auuauguuga gagcauauuu ccuccaaacc ucacagcaac |
| | | ucaaacaacu ggaaucugaa ggcaggaguc caauuuucac ucaucuuguu |
| | | acaagcuuaa aaggacuaug gacacuucgu gccuucggac ggcagccuua |
| | | cuuugaaacu cuguuccaca aagcucugaa uuuacauacu gccaacuggu |
| | | ucuuguaccu gucaacacug cgcugguucc aaaugagaau agaaaugauu |
| | | uuugucaucu ucuucauugc uguuaccuuc auuuccauuu uaacaacagg |
| | | agaaggagaa ggaagaguug guauuauccu gacuuuagcc augaauauca |
| | | ugaguacauu gcagugggcu guaaacucca gcauagaugu ggauagcuug |
| | | augcgaucug ugagccgagu cuuuaaguuc auugacaugc caacagaagg |
| | | uaaaccuacc aagucaacca aaccauacaa gaauggccaa cucucgaaag |
| | | uuaugauuau ugagaauuca cacgugaaga aagaugacau cuggcccuca |
| | | gggggccaaa ugacugucaa agaucucaca gcaaaauaca cagaaggugg |
| | | aaaugccaua uuagagaaca uuuccuucuc aauaaguccu ggccagaggu |
| | | ugacuuguca ucuugauuuc uggagaccac aagguaauga aaaauaauua |
| | | caagagucuu ccaucuguug caguauuaaa auggugggcc ucuugggaag |
| | | aacuggauca gggaagagua cuuuguuauc agcuuuuuug agacuacuga |
| | | acacugaagg agaaauccag aucgauggug ugucuuggga uucaauaacu |
| | | uugcaacagu ggaggaaagc cuuuggagug auaccacaga aaguauuuau |
| | | uuuuucugga acauuuagaa aaaacuugga ucccuaugaa caguggagug |
| | | aucaagaaau auggaaaguu gcagaugagg uugggcucag aucugugaua |
| | | gaacaguuuc cugggaagcu ugacuuuguc cuuguggaug ggggcugugu |
| | | ccuaagccau ggccacaagc aguugaugug cuuggcuaga ucuguucuca |
| | | guaaggcgaa gaucuugcug cuugaugaac ccagugcuca uuuggaucca |
| | | guaacauacc aaauaauuag aagaacucua aaacaagcau uugcugauug |
| | | cacaguaauu cucugugaac acaggauaga agcaaugcug gaaugccaac |
| | | aauuuuuggu cauagaagag aacaaagugc ggcaguacga uuccauccag |
| | | aaacugcuga acgagaggag ccucuuccgg caagccauca gccccuccga |
| | | cagggugaag cucuuucccc accggaacuc aagcaagugc aagucuaagc |
| | | cccagauugc ugcucugaaa gaggagacag aagaagaggu gcaagauaca |
| | | aggcuuuaga gagcagcaua aauguugaca ugggacauuu gcucauggaa |
| | | uuggagcucg ugggacaguc accucaugga auuggagcuc guggaacagu |
| | | uaccucugcc ucagaaaaca aggaugaauu aaguuuuuuu uuaaaaaaga |
| | | aacauuuggu aaggggaauu gaggacacug auaugggucu ugauaaaugg |
| | | cuuccuggca auagucaaau ugugugaaag guacuucaaa uccuugaaga |
| | | uuuaccacuu guguuuugca agccagauuu uccugaaaac ccuugccaug |
| | | ugcuaguaau uggaaaggca gcucuaaaug ucaaucagcc uaguugauca |
| | | gcuuauuguc uagugaaacu cguuaauuug uaguguugga gaagaacuga |
| | | aaucauacuu cuuaggguua ugauuaagua augauaacug gaaacuucag |
| | | cgguuuauau aagcuuguau uccuuuuucu cuccucuccc caugauguuu |
| | | agaaacacaa cuauauuguu ugcuaagcau uccaacuauc ucauuuccaa |
| | | gcaaguauua gaauaccaca ggaaccacaa gacugcacau caaaauaugc |
| | | cccauucaac aucuagugag cagucaggaa agagaacuuc cagauccugg |
| | | aaaucagggu uaguauuguc caggucuacc aaaaaucuca auauuucaga |
| | | uaaucacaau acaucccuua ccugggaaag ggcuguuaua aucuuucaca |
| | | ggggacagga ugguucccuu gaugaagaag uugauaugcc uuuucccaac |
| | | uccagaaagu gacaagcuca cagaccuuug aacuagaguu uagcuggaaa |
| | | aguauguuag ugcaaauugu cacaggacag cccuucuuuc cacagaagcu |
| | | ccagguagag gguguguaag uagauaggcc augggcacug uggguagaca |
| | | cacaugaagu ccaagcauuu agauguauag guugauggug guauguuuuc |
| | | aggcuagaug uauguacuuc augcugucua cacuaagaga gaaugagaga |
| | | cacacugaag aagcaccaau caugaauuag uuuuauaugc uucuguuuua |
| | | uaauuuugug aagcaaaauu uuuucucuag gaaauauuua uuuuaauaau |
| | | guuucaaaca uauauaacaa ugcuguauuu uaaaagaaug auuaugaauu |
| | | acauuuguau aaaauaauuu uuauauuuga aauauugacu uuuuauggca |
| | | cuaguauuuc uaugaaauau uauguuaaaa cugggacagg ggagaaccua |
| | | gggugauauu aaccaggggc caugaaucac cuuuuggucu ggagggaagc |
| | | cuuggggcug augcaguugu ugcccacagc uguaugauuc ccagccagca |
| | | cagccucuua gaugcaguuc ugaagaagau gguaccacca gucugacugu |
| | | uuccaucaag gguacacugc cuucucaacu ccaaacugac ucuuaagaag |
| | | acugcauuau auuuauuacu guaagaaaau aucacuuguc aauaaaaucc |
| | | auacauuugu gugaaa |
| Target sequence for | 37 | AAGCAGCATATTCTCAATACTATGTTTCATTAATAATTAAT |
| ASOs * (Bold - cryptic | | AGAGATATATGAACACATAAAAGATTCAATTATAATCACC |
| exon) | | TTGTGGATCTAAATTTCAGTTGACTTGTCATCTTGATTTC |
| | | TGGAGACCACAAGGTAATGAAAAATAATTACAAGAGTC |
| | | TTCCATCTGTTGCAGTATTAAAATGGTGAGTAAGACACC |
| | | CTGAAAGGAAATGTTCTATTCATGGTACAATGCAATTACAG |
| | | CTAGCACCAAATTCAACACTGTTTAACTTTCAACATATTAT |
| | | TTTG |
| Sequence motif no. 1 | 38 | GAUGGAAGA |
| Sequence motif no. 2 | 39 | CAACAGAUGGAAGA |

Corresponding to positions 17279930-117280013 in the genome according to the Assemble version used as updated in 2013 (UCSC Genome Browser on Human Dec. 2013 (GRCh38/hg38)).

### Materials and Methods

### Ussing Chamber Studies

Following differentiation HBE cells were analyzed for CFTR channel using the Ussing chamber system as previously described (Pranke et al., 2017). In general, the short-circuit-current (Isc) was measured under voltage clamp conditions with an EVC4000 Precision V/I Clamp (World Precision Instruments). For all measurements, chloride concentration gradient across the epithelium was applied by differential composition of basal and apical Ringer solutions. Inhibitors and activators were added after stabilization of baseline Isc: sodium(Na+)-channel blocker Amiloride (100 μM) to inhibit apical epithelial Na+channel (ENaC); cAMP agonist Forskolin (FSK, 10 μM) and 3-isobutyl-1-methylxanthine (IBMX 100μM) to activate the trans epithelial cAMP-dependent current; Genistein (10 μM) to potentiate the CFTR channel, CFTRinh172 (10 μM) -CFTR inhibitor to specifically inhibit CFTR and ATP (100 μM) to challenge the purinergic calcium-dependent Cl secretion. The following parameter was then calculated: ΔIsc CFTRinh172 as the difference between Isc after CFTR inh172 and Isc after FSK+Genistein.

### Example 1

### Treatment with SPL84-23-1 & Trikafta® Completely Restores CFTR Function in Heterozygous Patient-Derived Cells

The effect of TRIKAFATA® (elexacaftor/tezacaftor/ivacaftor), SPL84-23-1, and the combination of these two products on CFTR function in HBE cells was tested by the Ussing Chamber assay. The activity of CFTR following treatment with DMSO (control), TRIKAFATA®, SPL84-23-1, and a combination of TRIKAFATA® and SPL84-23-1, was calculated from the absolute values of ΔIscCFTRinh172(μA/cm²). Well differentiated primary HBE cells from a patient heterozygous for the 3849+10 kb C-to-T and F508del mutations were treated with 200 nM of SPL84-23-1. 48 hours before experiments, 3 μM VX661+1 μM VX-445 and 100 nM VX770 were added to the Trikafta®-treated filters. The level of WT was set according to the median ΔIscCFTRinh172 in HBE cultures from healthy WT/WT individuals from a previous study.

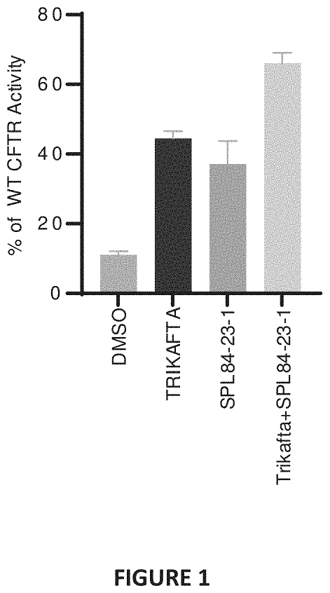

The data demonstrate that TRIKAFATA® and SPL84-23-1 alone each restored ˜40% of WT activity in HNEs from a patient that is heterozygous for the F508del and the 3849 mutations, while the SPL84-23/Trikafta® combination restores CFTR function to ˜70% of WT activity (FIG. 1).

### Example 2

### Treatment with SPL84-23-1 Completely Restores CFTR Function in Homozygous Patient-Derived Cells

The effect of SYMDEKO® (a combination of tezacaftor and ivacaftor) and SPL84-23-1 on CFTR function in HBE cells was tested using the Ussing Chamber assay. The activity of CFTR following the different treatments was calculated from the absolute values of ΔIscCFTRinh172(μA/cm2). Well differentiated primary HBE cells from a patient homozygous for the 3849+10 kb C-to-T mutation were treated with the 200 nM of SPL84-23-1. 48 hours before experiments, 3 μM VX661+100 nM VX770 were added to the Symdeko®-treated filters. The level of WT was set according to the median ΔIscCFTRinh172 in HBE cultures from healthy WT/WT individuals from a previous study.

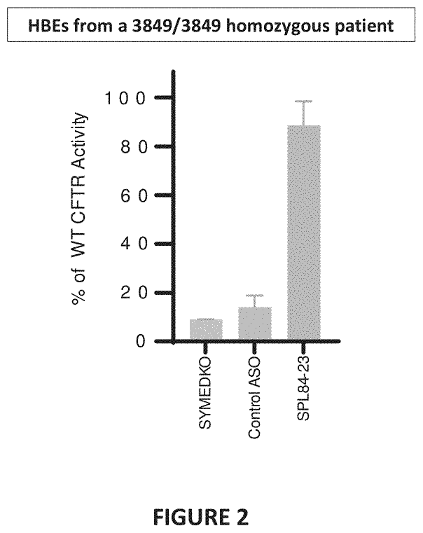

The data demonstrated that treatment with SPL84-23-1 completely (90% of WT) restored CFTR function in 3849 mutation homozygous patient derived cells. In contrast, SYMDEKO® did not restore CFTR activity in 3849 mutation homozygous patient derived cells (FIG. 2).

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

What is claimed is:

1. A method for treating Cystic Fibrosis (CF) in a subject heterozygous for the 3849+10 Kb C-to-T mutation in the CFTR gene, comprising administering to said subject a composition comprising a therapeutically effective amount of a synthetic oligonucleotide complementary to a region of the CFTR comprising the 3849+10 Kb C-to-T mutation and a composition comprising a therapeutically effective amount of one or more CFTR modifiers, wherein said synthetic oligonucleotide suppresses the inclusion of intron 22 cryptic exon in the mature CFTR mRNA.

2. The method of claim 1, wherein said subject comprises a 3849+10 Kb C-to-T mutation in one allele and a F508del mutation in a second allele of the CFTR gene.

3. The method of claim 1, wherein said one or more CFTR modifiers comprises a CFTR-splicing-modulating agent, Translational Read-Through agent, a CFTR amplifier, a CFTR potentiator, or a CFTR corrector.

4. The method of claim 1, wherein said one or more CFTR modifiers comprises a different synthetic oligonucleotide molecule capable of suppressing intron 22 cryptic exon inclusion in the mature CFTR mRNA, Ataluren, ELX-02, QBW251, PTI-808, VX-561, VX-121, ivacaftor (VX-770), lumacaftor (VX-809), tezacaftor (VX-661), elexacaftor (VX-445), VX-659, VX-152, VX-440, ABBV-2222 (formerly GLPG2222), ABBV-191, ABBV-3067, ABBV-3221 (formerly GLPG-3221), FDL169, PTI-801, PTI-428, or a combination thereof.

5. The method of claim 4, wherein said composition comprising one or more CFTR modifiers comprises elexacaftor, tezacaftor, and ivacaftor.

6. The method of claim 1, wherein said synthetic oligonucleotide comprises: a phosphate-ribose backbone, a phosphate-deoxyribose backbone, a phosphorothioate-deoxyribose backbone, a 2′-O-methyl-phosphorothioate (2′OMP) backbone, a phosphorodiamidate morpholino backbone, a peptide nucleic acid backbone, a 2-methoxyethyl phosphorothioate backbone, an alternating locked nucleic acid backbone, a phosphorothioate backbone, N3′-P5′ phosphoroamidates, 2′-deoxy-2′-fluoro-β-d-arabino nucleic acid, cyclohexene nucleic acid backbone nucleic acid, tricyclo-DNA (tcDNA) nucleic acid backbone, or a combination thereof.

7. The method of claim 1, wherein said synthetic oligonucleotide comprises a backbone with a 2′-Methoxy Ethyl (2′MOE) modification.

8. The method of claim 1, wherein the nucleotide sequence of said region of the CFTR comprising the 3849+10 Kb C-to-T mutation comprises SEQ ID NO: 37.

9. The method of claim 1, wherein said synthetic oligonucleotide molecule comprises a nucleotide sequence set forth in one of SEQ ID NOs: 1-25, and 41-44.

10. The method of claim 1, wherein the nucleotide sequence of said synthetic oligonucleotide molecule comprises the sequence as set forth in SEQ ID NO: 40.

11. The method of claim 1, wherein said treating comprises improving at least one clinical parameter of CF selected from the group consisting of: lung function, time to the first pulmonary exacerbation, change in weight, change in height, a change in Body Mass Index (BMI), change in the concentration of sweat chloride, number and/or duration of pulmonary exacerbations, total number of days of hospitalization for pulmonary exacerbations, and the need for antibiotic therapy for sinopulmonary signs or symptoms.

12. The method of claim 1, wherein said composition is administered via oral, nasal, inhalation, abdominal, subcutaneous, intra-peritoneal or intravenous administration.


SEQUENCE LISTING (71 sequences)

SEQ ID NO: 1 - RNA, 20 nt, Artificial Sequence
  Synthetic
cugcaacagauggaagacuc

SEQ ID NO: 2 - RNA, 19 nt, Artificial Sequence
  Synthetic
caacagauggaagacucuu

SEQ ID NO: 3 - RNA, 19 nt, Artificial Sequence
  Synthetic
cuccagaaaucaagaugac

SEQ ID NO: 4 - RNA, 19 nt, Artificial Sequence
  Synthetic
uacugcaacagauggaaga

SEQ ID NO: 5 - RNA, 20 nt, Artificial Sequence
  Synthetic
aucaagaugacaagucaacu

SEQ ID NO: 6 - RNA, 19 nt, Artificial Sequence
  Synthetic
guggucuccagaaaucaag

SEQ ID NO: 7 - RNA, 19 nt, Artificial Sequence
  Synthetic
gauggaagacucuuguaau

SEQ ID NO: 8 - RNA, 21 nt, Artificial Sequence
  Synthetic
caagaugacaagucaacugaa

SEQ ID NO: 9 - RNA, 22 nt, Artificial Sequence
  Synthetic
gaaaucaagaugacaagucaac

SEQ ID NO: 10 - RNA, 19 nt, Artificial Sequence
  Synthetic
accuuguggucuccagaaa

SEQ ID NO: 11 - RNA, 20 nt, Artificial Sequence
  Synthetic
ccagaaaucaagaugacaag

SEQ ID NO: 12 - RNA, 21 nt, Artificial Sequence
  Synthetic
caccauuuuaauacugcaaca

SEQ ID NO: 13 - RNA, 21 nt, Artificial Sequence
  Synthetic
uggaagacucuuguaauuauu

SEQ ID NO: 14 - RNA, 21 nt, Artificial Sequence
  Synthetic
gaugacaagucaacugaaauu

SEQ ID NO: 15 - RNA, 19 nt, Artificial Sequence
  Synthetic
cuuucagggugucuuacuc

SEQ ID NO: 16 - RNA, 19 nt, Artificial Sequence
  Synthetic
ucagggugucuuacucacc

SEQ ID NO: 17 - RNA, 20 nt, Artificial Sequence
  Synthetic
auuaccuuguggucuccaga

SEQ ID NO: 18 - RNA, 21 nt, Artificial Sequence
  Synthetic
gacaagucaacugaaauuuag

SEQ ID NO: 19 - RNA, 21 nt, Artificial Sequence
  Synthetic
caacugaaauuuagauccaca

SEQ ID NO: 20 - RNA, 21 nt, Artificial Sequence
  Synthetic
agucaacugaaauuuagaucc

SEQ ID NO: 21 - RNA, 21 nt, Artificial Sequence
  Synthetic
gugucuuacucaccauuuuaa

SEQ ID NO: 22 - RNA, 18 nt, Artificial Sequence
  Synthetic
gugucuuacucaccauuu

SEQ ID NO: 23 - RNA, 19 nt, Artificial Sequence
  Synthetic
cucaccauuuuaauacugc

SEQ ID NO: 24 - RNA, 18 nt, Artificial Sequence
  Synthetic
gacaagucaacugaaauu

SEQ ID NO: 25 - RNA, 20 nt, Artificial Sequence
  Synthetic
cuuacucaccauuuuaauac

SEQ ID NO: 26 - RNA, 18 nt, Artificial Sequence
  Synthetic
gucuuacucaccauuuua

SEQ ID NO: 27 - RNA, 19 nt, Artificial Sequence
  Synthetic
caagucaacugaaauuuag

SEQ ID NO: 28 - RNA, 19 nt, Artificial Sequence
  Synthetic
cuuguaauuauuuuuacau

SEQ ID NO: 29 - RNA, 25 nt, Artificial Sequence
  Synthetic
aaaucaagaugacaagucaacugaa

SEQ ID NO: 30 - RNA, 25 nt, Artificial Sequence
  Synthetic
cuuguggucuccagaaaucaagaug

SEQ ID NO: 31 - RNA, 25 nt, Artificial Sequence
  Synthetic
aacagauggaagacucuuguaauua

SEQ ID NO: 32 - RNA, 25 nt, Artificial Sequence
  Synthetic
ucagggugucuuacucaccauuuua

SEQ ID NO: 33 - RNA, 18 nt, Artificial Sequence
  Synthetic
gaccacuugccacccauc

SEQ ID NO: 34 - RNA, 6130 nt, Homo sapiens
aauuggaagcaaaugacaucacagcaggucagagaaaaaggguugagcggcaggcacccagaguaguaggucuuuggcauuaggagcuugagcccagacggcccuagcagggaccccagcgcccgagagaccaugcagaggucgccucuggaaaaggccagcguugucuccaaacuuuuuuucagcuggaccagaccaauuuugaggaaaggauacagacagcgccuggaauugucagacauauaccaaaucccuucuguugauucugcugacaaucuaucugaaaaauugaaagagaaugggauagagagcuggcuucaaagaaaaauccuaaacucauuaaugcccuucggcgauguuuuuucuggagauuuauguucuauggaaucuuuuuauauuuaggggaagucaccaaagcaguacagccucucuuacugggaagaaucauagcuuccuaugacccggauaacaaggaggaacgcucuaucgcgauuuaucuaggcauaggcuuaugccuucucuuuauugugaggacacugcuccuacacccagccauuuuuggccuucaucacauuggaaugcagaugagaauagcuauguuuaguuugauuuauaagaagacuuuaaagcugucaagccguguucuagauaaaauaaguauuggacaacuuguuagucuccuuuccaacaaccugaacaaauuugaugaaggacuugcauuggcacauuucguguggaucgcuccuuugcaaguggcacuccucauggggcuaaucugggaguuguuacaggcgucugccuucuguggacuugguuuccugauaguccuugcccuuuuucaggcugggcuagggagaaugaugaugaaguacagagaucagagagcugggaagaucagugaaagacuugugauuaccucagaaaugauugaaaauauccaaucuguuaaggcauacugcugggaagaagcaauggaaaaaaugauugaaaacuuaagacaaacagaacugaaacugacucggaaggcagccuaugugagauacuucaauagcucagccuucuucuucucaggguucuuugugguguuuuuaucugugcuucccuaugcacuaaucaaaggaaucauccuccggaaaauauucaccaccaucucauucugcauuguucugcgcauggcggucacucggcaauuucccugggcuguacaaacaugguaugacucucuuggagcaauaaacaaaauacaggauuucuuacaaaagcaagaauauaagacauuggaauauaacuuaacgacuacagaaguagugauggagaauguaacagccuucugggaggagggauuuggggaauuauuugagaaagcaaaacaaaacaauaacaauagaaaaacuucuaauggugaugacagccucuucuucaguaauuucucacuucuugguacuccuguccugaaagauauuaauuucaagauagaaagaggacaguuguuggcgguugcuggauccacuggagcaggcaagacuucacuucuaauggugauuaugggagaacuggagccuucagaggguaaaauuaagcacaguggaagaauuucauucuguucucaguuuuccuggauuaugccuggcaccauuaaagaaaauaucaucuuugguguuuccuaugaugaauauagauacagaagcgucaucaaagcaugccaacuagaagaggacaucuccaaguuugcagagaaagacaauauaguucuuggagaagguggaaucacacugaguggaggucaacgagcaagaauuucuuuagcaagagcaguauacaaagaugcugauuuguauuuauuagacucuccuuuuggauaccuagauguuuuaacagaaaaagaaauauuugaaagcugugucuguaaacugauggcuaacaaaacuaggauuuuggucacuucuaaaauggaacauuuaaagaaagcugacaaaauauuaauuuugcaugaagguagcagcuauuuuuaugggacauuuucagaacuccaaaaucuacagccagacuuuagcucaaaacucaugggaugugauucuuucgaccaauuuagugcagaaagaagaaauucaauccuaacugagaccuuacaccguuucucauuagaaggagaugcuccugucuccuggacagaaacaaaaaaacaaucuuuuaaacagacuggagaguuuggggaaaaaaggaagaauucuauucucaauccaaucaacucuauacgaaaauuuuccauugugcaaaagacucccuuacaaaugaauggcaucgaagaggauucugaugagccuuuagagagaaggcuguccuuaguaccagauucugagcagggagaggcgauacugccucgcaucagcgugaucagcacuggccccacgcuucaggcacgaaggaggcagucuguccugaaccugaugacacacucaguuaaccaaggucagaacauucaccgaaagacaacagcauccacacgaaaagugucacuggccccucaggcaaacuugacugaacuggauauauauucaagaagguuaucucaagaaacuggcuuggaaauaagugaagaaauuaacgaagaagacuuaaaggagugcuuuuuugaugauauggagagcauaccagcagugacuacauggaacacauaccuucgauauauuacuguccacaagagcuuaauuuuugugcuaauuuggugcuuaguaauuuuucuggcagagguggcugcuucuuugguugugcuguggcuccuuggaaacacuccucuucaagacaaagggaauaguacucauaguagaaauaacagcuaugcagugauuaucaccagcaccaguucguauuauguguuuuacauuuacgugggaguagccgacacuuugcuugcuaugggauucuucagaggucuaccacuggugcauacucuaaucacagugucgaaaauuuuacaccacaaaauguuacauucuguucuucaagcaccuaugucaacccucaacacguugaaagcaggugggauucuuaauagauucuccaaagauauagcaauuuuggaugaccuucugccucuuaccauauuugacuucauccaguuguuauuaauugugauuggagcuauagcaguugucgcaguuuuacaacccuacaucuuuguugcaacagugccagugauaguggcuuuuauuauguugagagcauauuuccuccaaaccucacagcaacucaaacaacuggaaucugaaggcaggaguccaauuuucacucaucuuguuacaagcuuaaaaggacuauggacacuucgugccuucggacggcagccuuacuuugaaacucuguuccacaaagcucugaauuuacauacugccaacugguucuuguaccugucaacacugcgcugguuccaaaugagaauagaaaugauuuuugucaucuucuucauugcuguuaccuucauuuccauuuuaacaacaggagaaggagaaggaagaguugguauuauccugacuuuagccaugaauaucaugaguacauugcagugggcuguaaacuccagcauagauguggauagcuugaugcgaucugugagccgagucuuuaaguucauugacaugccaacagaagguaaaccuaccaagucaaccaaaccauacaagaauggccaacucucgaaaguuaugauuauugagaauucacacgugaagaaagaugacaucuggcccucagggggccaaaugacugucaaagaucucacagcaaaauacacagaagguggaaaugccauauuagagaacauuuccuucucaauaaguccuggccagagggugggccucuugggaagaacuggaucagggaagaguacuuuguuaucagcuuuuuugagacuacugaacacugaaggagaaauccagaucgauggugugucuugggauucaauaacuuugcaacaguggaggaaagccuuuggagugauaccacagaaaguauuuauuuuuucuggaacauuuagaaaaaacuuggaucccuaugaacaguggagugaucaagaaauauggaaaguugcagaugagguugggcucagaucugugauagaacaguuuccugggaagcuugacuuuguccuuguggaugggggcuguguccuaagccauggccacaagcaguugaugugcuuggcuagaucuguucucaguaaggcgaagaucuugcugcuugaugaacccagugcucauuuggauccaguaacauaccaaauaauuagaagaacucuaaaacaagcauuugcugauugcacaguaauucucugugaacacaggauagaagcaaugcuggaaugccaacaauuuuuggucauagaagagaacaaagugcggcaguacgauuccauccagaaacugcugaacgagaggagccucuuccggcaagccaucagccccuccgacagggugaagcucuuuccccaccggaacucaagcaagugcaagucuaagccccagauugcugcucugaaagaggagacagaagaagaggugcaagauacaaggcuuuagagagcagcauaaauguugacaugggacauuugcucauggaauuggagcucgugggacagucaccucauggaauuggagcucguggaacaguuaccucugccucagaaaacaaggaugaauuaaguuuuuuuuuaaaaaagaaacauuugguaagggaauugaggacacugauaugggucuugauaaauggcuuccuggcaauagucaaauugugugaaagguacuucaaauccuugaagauuuaccacuuguguuuugcaagccagauuuuccugaaaacccuugccaugugcuaguaauuggaaaggcagcucuaaaugucaaucagccuaguugaucagcuuauugucuagugaaacucguuaauuuguaguguuggagaagaacugaaaucauacuucuuaggguuaugauuaaguaaugauaacuggaaacuucagcgguuuauauaagcuuguauuccuuuuucucuccucuccccaugauguuuagaaacacaacuauauuguuugcuaagcauuccaacuaucucauuuccaagcaaguauuagaauaccacaggaaccacaagacugcacaucaaaauaugccccauucaacaucuagugagcagucaggaaagagaacuuccagauccuggaaaucaggguuaguauuguccaggucuaccaaaaaucucaauauuucagauaaucacaauacaucccuuaccugggaaagggcuguuauaaucuuucacaggggacaggaugguucccuugaugaagaaguugauaugccuuuucccaacuccagaaagugacaagcucacagaccuuugaacuagaguuuagcuggaaaaguauguuagugcaaauugucacaggacagcccuucuuuccacagaagcuccagguagaggguguguaaguagauaggccaugggcacuguggguagacacacaugaaguccaagcauuuagauguauagguugauggugguauguuuucaggcuagauguauguacuucaugcugucuacacuaagagagaaugagagacacacugaagaagcaccaaucaugaauuaguuuuauaugcuucuguuuuauaauuuugugaagcaaaauuuuuucucuaggaaauauuuauuuuaauaauguuucaaacauauauaacaaugcuguauuuuaaaagaaugauuaugaauuacauuuguauaaaauaauuuuuauauuugaaauauugacuuuuuauggcacuaguauuucuaugaaauauuauguuaaaacugggacaggggagaaccuagggugauauuaaccaggggccaugaaucaccuuuuggucuggagggaagccuuggggcugaugcaguuguugcccacagcuguaugauucccagccagcacagccucuuagaugcaguucugaagaagaugguaccaccagucugacuguuuccaucaaggguacacugccuucucaacuccaaacugacucuuaagaagacugcauuauauuuauuacuguaagaaaauaucacuugucaauaaaauccauacauuugugugaaa

SEQ ID NO: 35 - DNA, 84 bp, Homo sapiens
ttgacttgtcatcttgatttctggagaccacaaggtaatgaaaaataattacaagagtcttccatctgttgcagtattaaaatg

SEQ ID NO: 36 - RNA, 6216 nt, Homo sapiens
aauuggaagcaaaugacaucacagcaggucagagaaaaaggguugagcggcaggcacccagaguaguaggucuuuggcauuaggagcuugagcccagacggcccuagcagggaccccagcgcccgagagaccaugcagaggucgccucuggaaaaggccagcguugucuccaaacuuuuuuucagcuggaccagaccaauuuugaggaaaggauacagacagcgccuggaauugucagacauauaccaaaucccuucuguugauucugcugacaaucuaucugaaaaauuggaaagagaaugggauagagagcuggcuucaaagaaaaauccuaaacucauuaaugcccuucggcgauguuuuuucuggagauuuauguucuauggaaucuuuuuauauuuaggggaagucaccaaagcaguacagccucucuuacugggaagaaucauagcuuccuaugacccggauaacaaggaggaacgcucuaucgcgauuuaucuaggcauaggcuuaugccuucucuuuauugugaggacacugcuccuacacccagccauuuuuggccuucaucacauuggaaugcagaugagaauagcuauguuuaguuugauuuauaagaagacuuuaaagcugucaagccguguucuagauaaaauaaguauuggacaacuuguuagucuccuuuccaacaaccugaacaaauuugaugaaggacuugcauuggcacauuucguguggaucgcuccuuugcaaguggcacuccucauggggcuaaucugggaguuguuacaggcgucugccuucuguggacuugguuuccugauaguccuugcccuuuuucaggcugggcuagggagaaugaugaugaaguacagagaucagagagcugggaagaucagugaaagacuugugauuaccucagaaaugauugaaaauauccaaucuguuaaggcauacugcugggaagaagcaauggaaaaaaugauugaaaacuuaagacaaacagaacugaaacugacucggaaggcagccuaugugagauacuucaauagcucagccuucuucuucucaggguucuuugugguguuuuuaucugugcuucccuaugcacuaaucaaaggaaucauccuccggaaaauauucaccaccaucucauucugcauuguucugcgcauggcggucacucggcaauuucccugggcuguacaaacaugguaugacucucuuggagcaauaaacaaaauacaggauuucuuacaaaagcaagaauauaagacauuggaauauaacuuaacgacuacagaaguagugauggagaauguaacagccuucugggaggagggauuuggggaauuauuugagaaagcaaaacaaaacaauaacaauagaaaaacuucuaauggugaugacagccucuucuucaguaauuucucacuucuugguacuccuguccugaaagauauuaauuucaagauagaaagaggacaguuguuggcgguugcuggauccacuggagcaggcaagacuucacuucuaauggugauuaugggagaacuggagccuucagaggguaaaauuaagcacaguggaagaauuucauucuguucucaguuuuccuggauuaugccuggcaccauuaaagaaaauaucaucuuugguguuuccuaugaugaauauagauacagaagcgucaucaaagcaugccaacuagaagaggacaucuccaaguuugcagagaaagacaauauaguucuuggagaagguggaaucacacugaguggaggucaacgagcaagaauuucuuuagcaagagcaguauacaaagaugcugauuuguauuuauuagacucuccuuuuggauaccuagauguuuuaacagaaaaagaaauauuugaaagcugugucuguaaacugauggcuaacaaaacuaggauuuuggucacuucuaaaauggaacauuuaaagaaagcugacaaaauauuaauuuugcaugaagguagcagcuauuuuuaugggacauuuucagaacuccaaaaucuacagccagacuuuagcucaaaacucaugggaugugauucuuucgaccaauuuagugcagaaagaagaaauucaauccuaacugagaccuuacaccguuucucauuagaaggagaugcuccugucuccuggacagaaacaaaaaaacaaucuuuuaaacagacuggagaguuuggggaaaaaaggaagaauucuauucucaauccaaucaacucuauacgaaaauuuuccauugugcaaaagacucccuuacaaaugaauggcaucgaagaggauucugaugagccuuuagagagaaggcuguccuuaguaccagauucugagcagggagaggcgauacugccucgcaucagcgugaucagcacuggccccacgcuucaggcacgaaggaggcagucuguccugaaccugaugacacacucaguuaaccaaggucagaacauucaccgaaagacaacagcauccacacgaaaagugucacuggccccucaggcaaacuugacugaacuggauauauauucaagaagguuaucucaagaaacuggcuuggaaauaagugaagaaauuaacgaagaagacuuaaaggagugcuuuuuugaugauauggagagcauaccagcagugacuacauggaacacauaccuucgauauauuacuguccacaagagcuuaauuuuugugcuaauuuggugcuuaguaauuuuucuggcagagguggcugcuucuuugguugugcuguggcuccuuggaaacacuccucuucaagacaaagggaauaguacucauaguagaaauaacagcuaugcagugauuaucaccagcaccaguucguauuauguguuuuacauuuacgugggaguagccgacacuuugcuugcuaugggauucuucagaggucuaccacuggugcauacucuaaucacagugucgaaaauuuuacaccacaaaauguuacauucuguucuucaagcaccuaugucaacccucaacacguugaaagcaggugggauucuuaauagauucuccaaagauauagcaauuuuggaugaccuucugccucuuaccauauuugacuucauccaguuguuauuaauugugauuggagcuauagcaguugucgcaguuuuacaacccuacaucuuuguugcaacagugccagugauaguggcuuuuauuauguugagagcauauuuccuccaaaccucacagcaacucaaacaacuggaaucugaaggcaggaguccaauuuucacucaucuuguuacaagcuuaaaaggacuauggacacuucgugccuucggacggcagccuuacuuugaaacucuguuccacaaagcucugaauuuacauacugccaacugguucuuguaccugucaacacugcgcugguuccaaaugagaauagaaaugauuuuugucaucuucuucauugcuguuaccuucauuuccauuuuaacaacaggagaaggagaaggaagaguugguauuauccugacuuuagccaugaauaucaugaguacauugcagugggcuguaaacuccagcauagauguggauagcuugaugcgaucugugagccgagucuuuaaguucauugacaugccaacagaagguaaaccuaccaagucaaccaaaccauacaagaauggccaacucucgaaaguuaugauuauugagaauucacacgugaagaaagaugacaucuggcccucagggggccaaaugacugucaaagaucucacagcaaaauacacagaagguggaaaugccauauuagagaacauuuccuucucaauaaguccuggccagagguugacuugucaucuugauuucuggagaccacaagguaaugaaaaauaauuacaagagucuuccaucuguugcaguauuaaaauggugggccucuugggaagaacuggaucagggaagaguacuuuguuaucagcuuuuuugagacuacugaacacugaaggagaaauccagaucgauggugugucuugggauucaauaacuuugcaacaguggaggaaagccuuuggagugauaccacagaaaguauuuauuuuuucuggaacauuuagaaaaaacuuggaucccuaugaacaguggagugaucaagaaauauggaaaguugcagaugagguugggcucagaucugugauagaacaguuuccugggaagcuugacuuuguccuuguggaugggggcuguguccuaagccauggccacaagcaguugaugugcuuggcuagaucuguucucaguaaggcgaagaucuugcugcuugaugaacccagugcucauuuggauccaguaacauaccaaauaauuagaagaacucuaaaacaagcauuugcugauugcacaguaauucucugugaacacaggauagaagcaaugcuggaaugccaacaauuuuuggucauagaagagaacaaagugcggcaguacgauuccauccagaaacugcugaacgagaggagccucuuccggcaagccaucagccccuccgacagggugaagcucuuuccccaccggaacucaagcaagugcaagucuaagccccagauugcugcucugaaagaggagacagaagaagaggugcaagauacaaggcuuuagagagcagcauaaauguugacaugggacauuugcucauggaauuggagcucgugggacagucaccucauggaauuggagcucguggaacaguuaccucugccucagaaaacaaggaugaauuaaguuuuuuuuuaaaaaagaaacauuugguaaggggaauugaggacacugauaugggucuugauaaauggcuuccuggcaauagucaaauugugugaaagguacuucaaauccuugaagauuuaccacuuguguuuugcaagccagauuuuccugaaaacccuugccaugugcuaguaauuggaaaggcagcucuaaaugucaaucagccuaguugaucagcuuauugucuagugaaacucguuaauuuguaguguuggagaagaacugaaaucauacuucuuaggguuaugauuaaguaaugauaacuggaaacuucagcgguuuauauaagcuuguauuccuuuuucucuccucuccccaugauguuuagaaacacaacuauauuguuugcuaagcauuccaacuaucucauuuccaagcaaguauuagaauaccacaggaaccacaagacugcacaucaaaauaugccccauucaacaucuagugagcagucaggaaagagaacuuccagauccuggaaaucaggguuaguauuguccaggucuaccaaaaaucucaauauuucagauaaucacaauacaucccuuaccugggaaagggcuguuauaaucuuucacaggggacaggaugguucccuugaugaagaaguugauaugccuuuucccaacuccagaaagugacaagcucacagaccuuugaacuagaguuuagcuggaaaaguauguuagugcaaauugucacaggacagcccuucuuuccacagaagcuccagguagaggguguguaaguagauaggccaugggcacuguggguagacacacaugaaguccaagcauuuagauguauagguugauggugguauguuuucaggcuagauguauguacuucaugcugucuacacuaagagagaaugagagacacacugaagaagcaccaaucaugaauuaguuuuauaugcuucuguuuuauaauuuugugaagcaaaauuuuuucucuaggaaauauuuauuuuaauaauguuucaaacauauauaacaaugcuguauuuuaaaagaaugauuaugaauuacauuuguauaaaauaauuuuuauauuugaaauauugacuuuuuauggcacuaguauuucuaugaaauauuauguuaaaacugggacaggggagaaccuagggugauauuaaccaggggccaugaaucaccuuuuggucuggagggaagccuuggggcugaugcaguuguugcccacagcuguaugauucccagccagcacagccucuuagaugcaguucugaagaagaugguaccaccagucugacuguuuccaucaaggguacacugccuucucaacuccaaacugacucuuaagaagacugcauuauauuuauuacuguaagaaaauaucacuugucaauaaaauccauacauuugugugaaa

SEQ ID NO: 37 - DNA, 284 bp, Homo sapiens
aagcagcatattctcaatactatgtttcattaataattaatagagatatatgaacacataaaagattcaattataatcaccttgtggatctaaatttcagttgacttgtcatcttgatttctggagaccacaaggtaatgaaaaataattacaagagtcttccatctgttgcagtattaaaatggtgagtaagacaccctgaaaggaaatgttctattcatggtacaatgcaattacagctagcaccaaattcaacactgtttaactttcaacatattattttg

SEQ ID NO: 38 - RNA, 9 nt, Artificial Sequence
  Synthetic
gauggaaga

SEQ ID NO: 39 - RNA, 14 nt, Artificial Sequence
  Synthetic
caacagauggaaga

SEQ ID NO: 40 - RNA, 19 nt, Artificial Sequence
  Synthetic
cugcaacagauggaagacu

SEQ ID NO: 41 - RNA, 19 nt, Artificial Sequence
  Synthetic
ugcaacagauggaagacuc

SEQ ID NO: 42 - RNA, 18 nt, Artificial Sequence
  Synthetic
cugcaacagauggaagac

SEQ ID NO: 43 - RNA, 18 nt, Artificial Sequence
  Synthetic
gcaacagauggaagacuc

SEQ ID NO: 44 - RNA, 18 nt, Artificial Sequence
  Synthetic
ugcaacagauggaagacu

SEQ ID NO: 45 - RNA, 17 nt, Artificial Sequence
  Synthetic
cugcaacagauggaaga

SEQ ID NO: 46 - RNA, 17 nt, Artificial Sequence
  Synthetic
caacagauggaagacuc

SEQ ID NO: 47 - RNA, 17 nt, Artificial Sequence
  Synthetic
ugcaacagauggaagac

SEQ ID NO: 48 - RNA, 17 nt, Artificial Sequence
  Synthetic
gcaacagauggaagacu

SEQ ID NO: 49 - RNA, 18 nt, Artificial Sequence
  Synthetic
cuccagaaaucaagauga

SEQ ID NO: 50 - RNA, 18 nt, Artificial Sequence
  Synthetic
uccagaaaucaagaugac

SEQ ID NO: 51 - RNA, 17 nt, Artificial Sequence
  Synthetic
cuccagaaaucaagaug

SEQ ID NO: 52 - RNA, 17 nt, Artificial Sequence
  Synthetic
ccagaaaucaagaugac

SEQ ID NO: 53 - RNA, 17 nt, Artificial Sequence
  Synthetic
uccagaaaucaagauga

SEQ ID NO: 54 - RNA, 18 nt, Artificial Sequence
  Synthetic
caacagauggaagacucu

SEQ ID NO: 55 - RNA, 18 nt, Artificial Sequence
  Synthetic
aacagauggaagacucuu

SEQ ID NO: 56 - RNA, 17 nt, Artificial Sequence
  Synthetic
acagauggaagacucuu

SEQ ID NO: 57 - RNA, 17 nt, Artificial Sequence
  Synthetic
aacagauggaagacucu

SEQ ID NO: 58 - RNA, 18 nt, Artificial Sequence
  Synthetic
uacugcaacagauggaag

SEQ ID NO: 59 - RNA, 18 nt, Artificial Sequence
  Synthetic
acugcaacagauggaaga

SEQ ID NO: 60 - RNA, 17 nt, Artificial Sequence
  Synthetic
uacugcaacagauggaa

SEQ ID NO: 61 - RNA, 17 nt, Artificial Sequence
  Synthetic
acugcaacagauggaag

SEQ ID NO: 62 - RNA, 19 nt, Artificial Sequence
  Synthetic
aucaagaugacaagucaac

SEQ ID NO: 63 - RNA, 19 nt, Artificial Sequence
  Synthetic
ucaagaugacaagucaacu

SEQ ID NO: 64 - RNA, 18 nt, Artificial Sequence
  Synthetic
aucaagaugacaagucaa

SEQ ID NO: 65 - RNA, 18 nt, Artificial Sequence
  Synthetic
caagaugacaagucaacu

SEQ ID NO: 66 - RNA, 18 nt, Artificial Sequence
  Synthetic
ucaagaugacaagucaac

SEQ ID NO: 67 - RNA, 17 nt, Artificial Sequence
  Synthetic
aucaagaugacaaguca

SEQ ID NO: 68 - RNA, 17 nt, Artificial Sequence
  Synthetic
aagaugacaagucaacu

SEQ ID NO: 69 - RNA, 17 nt, Artificial Sequence
  Synthetic
ucaagaugacaagucaa

SEQ ID NO: 70 - RNA, 17 nt, Artificial Sequence
  Synthetic
caagaugacaagucaac

SEQ ID NO: 71 - RNA, 20 nt, Artificial Sequence
  Synthetic
aauuauuuuucauuaccuug